(19) United States
(12) Patent Application Publication
(10) Pub. No.: US 2022/0273637 A1
(43) Pub. Date: Sep. 1, 2022

(54) NOVEL PFAR-INHIBITING COMPOUNDS

(71) Applicants: UNIVERSITE DE BRETAGNE OCCIDENTALE (UBO), BREST (FR); CENTRE NATIONAL DE LA RECHERCHE SCIENTIFIQUE, PARIS (FR); INSTITUT NATIONAL DE LA SANTE ET DE LA RECHERCHE MEDICALE, PARIS (FR); UNIVERSITE DE MONTPELLIER, MONTPELLIER (FR); UNIVERSITE DE STRASBOURG, STRASBOURG (FR); CENTRE HOSPITALIER RÉGIONAL ET UNIVERSITAIRE DE BREST, BREST (FR); SORBONNE UNIVERSITE, PARIS (FR); ETABLISSEMENT FRANCAIS DU SANG, LA PLAINE SAINT DENIS CEDEX (FR); ASSOCIATION INSTITUT DE MYOLOGIE, PARIS (FR); ASSOCIATION FRANÇAISE CONTRE LES MYOPATHIES, PARIS CEDEX 13 (FR)

(72) Inventors: MARTINE SIMONELIG, CLAPIERS (FR); FRÉDÉRIC BIHEL, FEGERSHEIM (FR); CÉCILE VOISSET, BREST (FR); CAPUCINE TROLLET, PARIS (FR)

(21) Appl. No.: 17/625,348

(22) PCT Filed: Jul. 9, 2020
(86) PCT No.: PCT/EP2020/069466

§ 371 Date: Jan. 7, 2022

(30) Foreign Application Priority Data
Jul. 9, 2019 (EP) 19315064.6
Oct. 2, 2019 (EP) 19201179.9

(51) Int. Cl. A61K 31/4515; A61K 31/55; A61K 31/381; A61K 45/06

(52) U.S. Cl. CPC A61K 31/4515; A61K 31/55; A61K 31/381; A61K 45/06

(57) ABSTRACT

The invention relates to novel compounds which are inhibitors of the ribosome protein chaperone activity (“protein folding activity of the ribosome” or “PFAR”). More particularly, the invention relates to their use as PFAR-inhibitors, to compositions comprising them and to methods for treating proteinopathies.

### TECHNICAL FIELD

The present invention belongs to the technical field of inhibitors of the protein chaperone activity of ribosome (protein folding activity of ribosome or PFAR). More particularly, the invention relates to PFAR inhibitors for their use in the treatment of diseases linked to the poor folding and/or aggregation of proteins.

### PRIOR ART

Most neurodegenerative diseases share the common feature of the presence of misfolded proteins that form intracellular or extracellular amyloid aggregates. They are then described as aggregopathies, proteinopathies, foldopathies, protein conformational disorders, or protein misfolding diseases. They encompass for example Creutzfeldt-Jakob disease, Alzheimer's disease or else Parkinson's disease. The presence of proteins that aggregate due to their structural conformation has since been identified in targets other than the central nervous system. The number of diseases found to correspond to proteinopathies is constantly increasing.

The concept of infectious proteins was initially established for the prion protein PrP in mammals with subacute transmissible spongiform encephalopathy. The prion protein PrP is found in two isoforms: PrP^{C} and PrP^{res}. PᵣPres ddiffers from PrP^{C} by its three-dimensional structure and its propensity to form amyloid fibers. In its PrP^{res} conformation, the prion protein accumulates in the form of amyloid fibers which propagate spontaneously in the absence of prion-specific nucleic acid. This accumulation leads to a degeneration of the central nervous system associated in particular with dementia and spongiosis. In animals, well-known examples of prion diseases are scrapie, bovine spongiform encephalopathy and chronic wasting disease of cervids. In humans, mention may be made of Creutzfeldt-Jakob disease, fatal familial insomnia and Gerstmann-Straussler-Scheinker syndrome.

Other proteins are known for accumulating in the form of aggregates in neurodegenerative diseases. In humans, Alzheimer's disease (accumulation of tau and beta-amyloid proteins), Parkinson's disease (accumulation of alpha-synuclein), amyotrophic lateral sclerosis (accumulation of Fus and TDP43), oculopharyngeal muscular dystrophy (accumulation of PABPN1) are a few examples thereof. Different on many levels, these proteins however have in common their propensity to change conformation and to form amyloid fibers.

The research initiated on prion diseases has shown the involvement of ribosome in the propagation of the pathological conformation of prion proteins. Ribosome, in addition to its activity in protein synthesis, is also involved in protein folding. This second activity, referred to as protein chaperone activity (protein folding activity of ribosome or PFAR), was associated with prion diseases for the first time in 2008, with two anti-prion molecules: 6-aminophenanthridine and guanabenz (Tribouillard-Tanvier et al. 2008). Later, imiquimod (Oumata et al. 2013), flunarizine and metixene (Nguyen 2013) were described as compounds that inhibit PFAR and are active in models of prion diseases. 6-Aminophenanthridine and guanabenz, PFAR inhibitors, also displayed an efficacy in other models of proteinopathies, such as for Huntington's disease (EP2066312B1) and for oculopharyngeal muscular dystrophy (Barbezier et al. 2011; Malerba et al. 2019). Guanabenz was also shown to be effective for reducing the toxicity of the aggregation of the TDP43 protein in a model for amyotrophic lateral sclerosis (Vaccaro et al. 2013).

Thus, the inhibition of PFAR appears to be a therapeutic target of choice for the treatment of proteinopathies. In the absence of treatment for these diseases, there is therefore an urgent need to identify new drug compounds.

### SUMMARY OF THE INVENTION

The invention meets this need by proposing one or more PFAR-inhibiting compounds for the use thereof in the treatment of a proteinopathy. The invention also relates to the use of a composition comprising one or more PFAR-inhibiting compounds, for the manufacture of a drug intended to treat a proteinopathy. The invention also targets a method for treating a proteinopathy, comprising the administration of an effective amount of one or more PFAR-inhibiting compounds, to a subject having need thereof. These compounds, the PFAR-inhibiting property of which is demonstrated for the first time by the inventors, are described hereinbelow.

Thus, one subject according to the invention relates to a composition comprising at least one compound chosen from ebastine, azelastine, duloxetine, atomoxetine, benzydamine, biperiden, chloropyramine, citalopram, dicyclomine, nefopam, orphenadrine, prenylamine, triflupromazine and zimelidine, or one of the pharmaceutically acceptable salts, hydrates, isomers and racemates thereof, for use thereof in the treatment of a proteinopathy. The invention also relates to the combinations of these compounds.

In particular, the invention relates to a composition comprising ebastine or a pharmaceutically acceptable salt, hydrate, isomer and racemate, for use thereof in the treatment of a proteinopathy.

As an alternative, the invention targets a composition comprising azelastine or a pharmaceutically acceptable salt, hydrate, isomer and racemate, for use thereof in the treatment of a proteinopathy.

As an alternative, the invention targets a composition comprising duloxetine or a pharmaceutically acceptable salt, hydrate, isomer and racemate, for use thereof in the treatment of a proteinopathy.

According to a preferred implementation, the composition according to the invention for use thereof in the treatment of a proteinopathy further comprises at least one different compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or the pharmaceutically acceptable salts, hydrates, isomers and racemates thereof.

According to one embodiment, the composition for use thereof in the treatment of a proteinopathy according to the invention may be chosen from the following combinations: ebastine and flunarizine, ebastine and azelastine, ebastine and loperamide, azelastine and flunarizine, azelastine and loperamide, and flunarizine and loperamide, or the pharmaceutically acceptable salts, hydrates, isomers and racemates thereof.

Preferably, the compounds intended to be used in combination according to the present invention may be administered simultaneously, separately or sequentially.

Moreover, the proteinopathy may be chosen from Creutzfeldt-Jakob disease, Gerstmann-Straussler-Scheinker syndrome, fatal familial insomnia, kuru, VPSPr disease, Lewy body disease, Parkinson's disease, Alzheimer's disease, Huntington's disease, amyotrophic lateral sclerosis, frontotemporal dementia, type 2 diabetes, oculopharyngeal muscular dystrophy, bovine spongiform encephalopathy, scrapie, chronic wasting disease of cervids, feline spongiform encephalopathy, camel spongiform encephalopathy and exotic ungulate encephalopathy.

According to a preferred implementation, said proteinopathy is linked to the accumulation of prion proteins PrP in the form of aggregates.

Alternatively, said proteinopathy is chosen from Creutzfeldt-Jakob disease, Gerstmann-Straussler-Scheinker syndrome, fatal familial insomnia, kuru and VPSPr disease.

Consequently, the invention also targets a composition according to the invention for use thereof in the treatment of Creutzfeldt-Jakob disease.

Moreover, the invention targets a composition comprising at least one compound chosen from ebastine, azelastine, duloxetine, atomoxetine, benzydamine, biperiden, chloropyramine, citalopram, dicyclomine, nefopam, orphenadrine, prenylamine, triflupromazine and zimelidine, or the pharmaceutically acceptable salts, hydrates, isomers and racemates thereof, in combination with at least one different compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or the pharmaceutically acceptable salts, hydrates, isomers and racemates thereof.

Moreover, the invention targets a composition comprising ebastine in combination with at least one different compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or the pharmaceutically acceptable salts, hydrates, isomers and racemates thereof.

Moreover, the invention targets a composition comprising at least one compound chosen from azelastine in combination with at least one different compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or the pharmaceutically acceptable salts, hydrates, isomers and racemates thereof.

Moreover, the invention targets a composition comprising duloxetine in combination with at least one different compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or the pharmaceutically acceptable salts, hydrates, isomers and racemates thereof.

According to one embodiment, the invention targets a composition or a composition for use thereof as described above, further comprising pharmaceutically acceptable carriers or excipients.

Moreover, the compounds according to the invention which are useful for the treatment of a proteinopathy are PFAR inhibitors. Consequently, the invention also targets at least one compound chosen from ebastine, alimemazine (trimeprazine), amitriptyline, astemizole, atomoxetine, azelastine, benzydamine, biperiden, chloropyramine, citalopram, clemastine, clomipramine, desipramine, desloratadine, dicyclomine, diphenhydramine, doxepin, duloxetine, fluoxetine, haloperidol, imipramine, loperamide, nefopam, orphenadrine, prenylamine, reboxetine, thioridazine, trifluoperazine, triflupromazine, chlorpromazine, quinacrine (mepacrine) and zimelidine, or the pharmaceutically acceptable salts, hydrates, isomers and racemates thereof, or any combination thereof, for inhibiting PFAR, in other words for inhibiting the protein chaperone activity. Preferably, said use is an in vitro or ex vivo use.

Definitions

In the context of the invention, the term “about” is used to indicate that a value includes the variations inherent to the margin of error linked to the use of a measurement device, to the method used to determine the value, or the variations that may exist between the cells within the population studied and between the populations. Thus, in one embodiment, the term “about”, placed in front of a value, corresponds to plus or minus 10% of this value.

In the context of the invention, the term “proteinopathy” or “proteinopathies” denotes a disease or a group of diseases also known under the terms “protein misfolding disease” or “conformational disorders” or “aggregopathy” or “foldopathy”. These diseases are characterized by the accumulation of proteins which have a three-dimensional structure different to that observed for the same protein in a healthy subject, in particular that are enriched in beta sheets and which form amyloid fibers, leading to the accumulation of protein aggregates. This accumulation may take place on the inside or on the outside of the cell nuclei, and on the inside or on the outside of the cell. It may occur in all types of cells, notably the cells of the central nervous system, neurones in general, muscle cells, β cells of the islets of Langerhans, and leads to cell death. A “neurological proteinopathy” denotes more specifically a disease or a group of diseases characterized by the accumulation of protein aggregates in the central nervous system.

Within the meaning of the invention, the expression “to inhibit PFAR” means the ability to reduce or completely prevent the action of ribosome on protein folding, notably by inhibiting the assisted folding activity borne by the ribosome, such as for example the renaturation of hCA or BCA II proteins assisted by whole ribosomes (70S) of E. coli or thedomain V of the large rRNA of the large subunit of the ribosome transcribed in vitro (Tribouillard-Tanvier et al. 2008b; Blonde) et al. 2016). The inhibition of PFAR can be evaluated according to the protocol described in the examples below (e.g. FIG. 1).

In the context of the invention, the designation of a drug or of a particular compound is considered to involve not only the named molecule, but also its pharmaceutically acceptable salts, hydrates, derivatives, isomers, racemates, conjugates, prodrugs and prodrug derivatives of any chemical purity.

The expression “pharmaceutically acceptable salt” refers to organic or inorganic salts, which retain the activity and the biological properties of the compounds of the invention, and which are relatively non-toxic. The pharmaceutical formation of salt consists of the pairing of an acidic, basic or zwitterionic molecule with a counterion to create the salt of the drug. Examples of pharmaceutically acceptable salts according to the invention include those obtained by reaction of the main compound, functioning as a base, with an organic or inorganic acid to form a salt, for example salts of acetic acid, nitric acid, tartaric acid, hydrochloric acid, sulfuric acid, phosphoric acid, methanesulfonic acid, camphorsulfonic acid, oxalic acid, maleic acid, succinic acid or citric acid. The pharmaceutically acceptable salts according to the invention also include those for which the main compound functions as an acid and reacts with a suitable base to form, for example, salts of sodium, potassium, calcium, magnesium, ammonium or choline.

By “hydrate”, it is meant a form of association or combination of a compound with one or more water molecules. For example, the forms may be named hemihydrate, dihydrate and trihydrate of a compound.

The term “derivative”, when it refers to a compound, includes any molecule which is functionally or structurally bound to said compound, such as an acid, an amide, an ester, an ether, an acetylated variant, a hydroxylated variant, an alkyl variant of such a compound. The term “derivative” also includes compounds which have one or more substituents that do not modify, at least substantially, the function of said compound (here the inhibition of PFAR).

The term “isomer” denotes here the stereoisomers, such as the enantiomers (e.g.

levorotatory, dextrorotatory), diastereomers and epimers and also conformational isomers. In the context of the present invention, it is understood that the isomer of a compound retains all or some of the function of said compound (here the inhibition of PFAR).

The expression “racemate”, “racemic” or “racemic mixture” denotes here a mixture, in equal proportions, of the levorotatory and dextrorotatory enantiomers of a chiral compound.

The term “conjugate” is understood to denote a compound comprising a succession of alternating single and double bonds which interact with one another and enable the delocalization of electrons. Therefore, a conjugated compound may be in several so-called Lewis forms (referred to as mesomeric, resonance or canonical forms).

The term “prodrug” is used in the present description to denote any functional derivative (or precursor) of a compound according to the invention which, when it is administered to a patient, provides said compound following a spontaneous chemical reaction, a chemical catalysis reaction, and/or a chemical metabolic reaction. Prodrugs typically have an X-drug structure, in which X is an inert carrier group and the drug is the active compound. The technical information that makes it possible to select a suitable prodrug is part of the general knowledge (Ettmayer et al. 2004; Beaumont et al. 2003; Heimbach et al. 2003; Yang et al. 1999; Steffansen et al. 2004). Furthermore, the preparation of prodrugs may be carried out by conventional methods known to those skilled in the art. Methods which may be used to synthesize prodrugs are described in the literature (Ettmayer et al. 2004; Stella 2007; Wermuth 2003; Pezron et al. 2002; Stella 2004; Stella & Nti-Addae 2007; Higuchi & Stella 1975).

According to a preferred embodiment, the designation of a compound corresponds to the designation of the compound as such, as well as any pharmaceutically acceptable salt, hydrate, isomer and racemate of said compound.

Within the meaning of the invention, “treatment” includes therapy, prevention, prophylaxis, delay or reduction of the symptoms caused by a protein misfolding disease. The term “treatment” denotes the reduction of the amount of misfolded proteins or the maintenance thereof at a relatively constant level. Thus, the term “treatment” denotes the reduction of the amount of protein aggregates or the maintenance thereof at a constant level. The term “treatment” includes in particular the control of the progression of the proteinopathy. More specifically, the term “treatment” includes the inhibition of PFAR in the subjects treated. Preferably, the treatment is administered to subjects who are animals or humans. In particular, the subjects treated are humans, bovids, camelids, cervids, felines, ungulates or ovines.

According to the invention, a “combination of compounds for use thereof in the treatment of a proteinopathy” denotes a treatment in which at least two drugs are administered together or separately, at the same time or sequentially. These at least two drugs may be administered by different routes or protocols, thus they may be formulated together or separately.

The expression “therapeutically effective amount” is understood to mean the amount of a compound alone or in combination according to the invention which is effective in the treatment of the proteinopathy. The therapeutically effective amount may be determined by the practitioner or those skilled in the art as a function of the size, age, general state of health of the patient, the disease specifically involved and its severity, the mode of administration and other relevant circumstances.

### BRIEF DESCRIPTION OF THE FIGURES

FIG. 1: Schematic representation of the experimental protocol used to identify the compounds capable of inhibiting PFAR in yeast cells. The yeasts of yeast Itv1Δhsp104A [psi-] (Blonde) et al. 2016) are pretreated with various concentrations of PFAR-inhibiting compounds or with dimethyl sulfoxide (DMSO, control treatment) for 2 hours. They then undergo a heat shock at 43.5° C. for one hour, which leads to the denaturation of the proteins. The protein synthesis is then inhibited by the addition of cycloheximide. In the presence of DMSO alone, luciferase regains correct folding owing to the PFAR activity of ribosome. On the other hand, when the compounds have the ability to inhibit PFAR, luciferase cannot recover its initial folding. In this way, the measurement of the activity of luciferase makes it possible to identify the PFAR-inhibiting activity of the compounds tested. DMSO: dimethyl sulfoxide.

FIG. 2: Effects of ebastine, astemizole, clemastine, metixene, triflupromazine, azelastine, duloxetine, loperamide, thioridazine and flunarizine on PFAR in the yeast model. Antazoline and diazepam, two molecules that are inactive against prions, are not capable of inhibiting PFAR and were used as controls. The measurements were carried out at the times 0 minute, 90 minutes and 150 minutes. In each of the tests, the use of DMSO shows that the luciferase activity changes from less than 40% of its initial value at 0 minute, to at least 70% at 90 minutes then to approximately 100% after 150 minutes. Thus, in 150 minutes and in the absence of inhibiting compounds, the PFAR activity of ribosome makes it possible to re-establish the functional conformation of the proteins. Diazepam and antazoline do not inhibit PFAR at relatively high concentrations (200 μM). On the other hand, the compounds tested all made it possible to inhibit PFAR: ebastine from 25 μM; astemizole and thioridazine from 50 μM; flunarizine, clemastine, metixene and triflupromazine from 100 μM; azelastine, duloxetine and loperamide from 150 μM. The experiments were repeated twice. The histograms represent the mean percentages including three technical repetitions; the error bars show the standard deviation at the mean. %LA: luciferase activity expressed as percentage; DMSO: dimethyl sulfoxide.

FIG. 3: Effects of astemizole, azelastine, duloxetine, ebastine, flunarizine, loperamide, metixene and triflupromazine against prions in a cell model (MovS6 cells). Flunarizine and also astemizole, azelastine, duloxetine, ebastine, loperamide, triflupromazine and metixene displayed good anti-PrP^{Sc} activity making it possible to reduce the PrP^{Sc} load with IC₅₀ <4 μM. None of the compounds had an influence on the PrP^{tot} load. These results demonstrate that the PFAR-inhibiting compounds are capable of reducing the propagation of the prion PrP^{Sc}. The blots presented are representative of two independent tests which gave similar results.

FIG. 4: Effects of azelastine, duloxetine, ebastine, flunarizine, loperamide, metixene and astemizole against prions in an organotypic model. Flunarizine and also astemizole, azelastine, duloxetine, ebastine, loperamide and metixene displayed good anti-PrP^{Sc} activity making it possible to reduce the PrP^{Sc} load to 64% (azelastine at 20 μM), 59% (duloxetine at 30 μM), 50% (ebastine at 30 μM), down to even 28% (loperamide at 30 μM). None of the compounds had an influence on the PrP^{tot} load. These results demonstrate that the PFAR-inhibiting compounds are good candidates for the treatment of proteinopathies. The blots presented are representative of two independent tests which gave similar results.

FIG. 5: Effects of the azelastine and flunarizine, ebastine and flunarizine, and ebastine and loperamide combinations against prions in a cell model of proteinopathy. On the immunoblots, the degree of accumulation of PrP^{Sc} is estimated by the ratio, as a percentage, of the PrP^{Sc} load to the tubulin load. Dimethyl sulfoxide alone (DMSO) is used to fix the reference value (100%) in the absence of the compounds to be tested. Azelastine, flunarizine, ebastine and loperamide were used at particularly low concentrations (respectively 1.5 μM, 3.5 μM, 3.5 μM and 0.75 μM), for which they display no or little efficacy when taken separately (respectively the PrP^{Sc}/tubulin ratio is 104%, 85%, 118% and 114%). At the same concentrations, flunarizine combined with azelastine or ebastine makes it possible to lower the degree of accumulation of PrP^{Sc} to 43%; the combination of ebastine and loperamide makes it possible to lower this degree to 65%. These values are surprising in that they are greater than the sum of the individual effects. The photograph of the immunoblot presented is representative of the results obtained during three independent experiments. AZE: azelastine; FLU: flunarizine; EBA: ebastine; LOP: loperamide; DMSO: dimethyl sulfoxide.

FIG. 6: Effects of flunarizine, metixene, thioridazine, astemizole, loperamide, duloxetine, triflupromazine, clemastine, azelastine and ebastine in a cell model of oculopharyngeal muscular dystrophy (OPMD). In the absence of treatment (DMSO), more than 40% of nuclei having aggregates of the nuclear PABPN1 protein are listed. All the compounds were tested at a concentration of 10 μM. Guanabenz was used as a positive control, whilst diazepam was used as a negative control. The percentage of nuclei having aggregates of the PABPN1 protein was significantly reduced by the compounds tested, in a dose-dependent manner. These results demonstrate that the PFAR-inhibiting compounds are effective in the treatment of proteinopathies such as OPMD. The differences with the DMSO treatment are statistically significant with a value of **p<0.01; ***p<0.001;****p<0.0001; DMSO: dimethyl sulfoxide.

FIG. 7: Effects of flunarizine, metixene, guanabenz and ebastine in an animal model of oculopharyngeal muscular dystrophy (OPMD). In the presence of DMSO used as control (1.5% or 2% according to the indications), the number of flies having an abnormal wing position increases after day 3. Flunarizine (top panel), metixene (middle panel) and also ebastine (bottom panel) make it possible to reduce in a statistically significant manner the percentage of flies that have an abnormal wing position. These results confirm that the PFAR-inhibiting compounds are effective in the treatment of proteinopathies such as OPMD. The differences with the DMSO treatment are statistically significant with a p value: *p<0.1; **p<0.01; ***p<0.001; DMSO: dimethyl sulfoxide.

### DETAILED DESCRIPTION

The invention relates to novel PFAR inhibitors, in particular the use thereof to inhibit PFAR, but also for the use thereof in the treatment of proteinopathies.

It has previously been demonstrated that flunarizine has abilities to inhibit PFAR and that it is effective against prions (Nguyen 2013). Thus, in a first study, the inventors carried out a screening of compounds according to a structure-activity approach on the basis of flunarizine. The compounds thus screened were then evaluated for their anti-prion potential and their ability to inhibit PFAR.

By means of this method, the inventors were able to identify compounds useful for inhibiting PFAR and which could be repositioned in the treatment of proteinopathies.

PFAR, protein folding activity borne by the ribosome, was described for the first time in vitro by the group of C. Das Gupta which demonstrated that denatured proteins can regain their functional conformation by means of the domain V of the large ribosomal RNA (rRNA) of the large subunit of the ribosome (Das et al. 2008). Ribosome-assisted folding was then described for the ribosomes of all kingdoms of life and for all classes and sources of proteins tested (Das et al. 2008; Barbezier et al. 2011), which is in keeping with the high conservation of the sequence and of the secondary structure of the domain V of the rRNA (Ben-Shem et al. 2011). PFAR involves the domain V of the large ribosomal RNA (rRNA) of the large subunit 60S of the ribosome, as has been demonstrated owing to a reverse screening approach for the identification of one of the cellular targets of 6AP and GA (Tribouillard-Tanvier et al. 2008a and 2008b). The domain V of the large rRNA (23S in E. coli, 25S in S. cerevisiae, 28S in Metazoa) is a ribozyme bearing 2 enzymatic activities: (i) a peptidyl transferase activity, and (ii) PFAR (Das et al. 2008; Voisset et al. 2008). The bond from 6AP and GA to the rRNA 23S specifically inhibits PFAR, without interfering with the translational activity of the ribosome (Tribouillard-Tanvier et al. 2008a and 2008b).

The nucleotides of the domains V of the rRNA 23S of E. coli and 25S of S. cerevisiae which are essential to the protein chaperone activity of ribosome in vitro have been identified (Pang et al. 2013). These studies demonstrated that most of the nucleotides involved in PFAR are conserved in the bacterium (23S) and in the yeast (25S) (Pang et al. 2013), and that the 6AP and GA are competitive inhibitors of PFAR (Reis et al. 2011).

It has recently been shown that PFAR is involved in the propagation of the prion [PSI+] in yeast (Blonde) et al. 2016).

Compounds and Compositions for use thereof in the Treatment of Proteinopathies

One subject according to the invention is a pharmaceutical composition comprising at least one PFAR-inhibiting compound for use thereof in the treatment of a proteinopathy. The invention also relates to the use of a composition comprising at least one PFAR-inhibiting compound, for the manufacture of a drug intended to treat a proteinopathy. The invention also targets a method for treating a proteinopathy, comprising the administration of an effective amount of at least one PFAR-inhibiting compound, to a subject having need thereof.

Indeed, as disclosed in the examples, the PFAR inhibitors have shown a beneficial activity in models for prion diseases and oculopharyngeal muscular dystrophy.

The compounds used according to the invention are described below. The PFAR-inhibiting property thereof is demonstrated here for the first time.

In particular, the composition for use thereof in the treatment of a proteinopathy comprises at least one compound chosen from ebastine, azelastine, duloxetine, atomoxetine, benzydamine, biperiden, chloropyramine, citalopram, dicyclomine, nefopam, orphenadrine, prenylamine, triflupromazine and zimelidine, or the pharmaceutically acceptable salts, hydrates, isomers and racemates thereof.

According to one embodiment, the composition for use thereof in the treatment of a proteinopathy comprises at least one compound chosen from ebastine, azelastine, duloxetine, atomoxetine, benzydamine, biperiden, chloropyramine, citalopram, dicyclomine, nefopam, orphenadrine, prenylamine, triflupromazine and zimelidine, in combination with at least one different compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or the pharmaceutically acceptable salts, hydrates, isomers and racemates thereof.

In particular, the composition for use thereof in the treatment of a proteinopathy comprises at least one compound chosen from ebastine, azelastine, duloxetine, atomoxetine, benzydamine, biperiden, chloropyramine, citalopram, dicyclomine, nefopam, orphenadrine, prenylamine, triflupromazine and zimelidine, in combination with flunarizine, or the pharmaceutically acceptable salts, hydrates, isomers and racemates thereof.

Preferably, the composition comprises ebastine, or one of the pharmaceutically acceptable salts, hydrates, isomers and racemates thereof, in combination with at least one compound chosen from flunarizine, loperamide, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof, for use thereof in the treatment of a proteinopathy.

According to one variant, the composition comprises azelastine, or one of the pharmaceutically acceptable salts, hydrates, isomers and racemates thereof, in combination with at least one compound chosen from flunarizine, loperamide, ebastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof, for use thereof in the treatment of a proteinopathy.

According to another variant, the composition comprises duloxetine, or one of the pharmaceutically acceptable salts, hydrates, isomers and racemates thereof, in combination with at least one compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof, for use thereof in the treatment of a proteinopathy.

More preferably, the composition for use thereof in the treatment of a proteinopathy comprises at least two compounds chosen from ebastine, azelastine, flunarizine, loperamide, astemizole, duloxetine, metixene, clemastine, thioridazine and triflupromazine, preferably from ebastine, flunarizine, loperamide and azelastine, or the pharmaceutically acceptable salts, hydrates, isomers and racemates thereof.

According to one embodiment, the composition for use thereof comprises ebastine, or one of the pharmaceutically acceptable salts, hydrates, isomers and racemates thereof, in combination with a compound chosen from azelastine, flunarizine, loperamide, astemizole, duloxetine, metixene, clemastine, thioridazine and triflupromazine, or one of the pharmaceutically acceptable salts, hydrates, isomers and racemates thereof.

According to one embodiment, the composition for use thereof comprises azelastine, or one of the pharmaceutically acceptable salts, hydrates, isomers and racemates thereof, in combination with a compound chosen from ebastine, flunarizine, loperamide, astemizole, duloxetine, metixene, clemastine, thioridazine and triflupromazine, or one of the pharmaceutically acceptable salts, hydrates, isomers and racemates thereof.

According to one embodiment, the composition for use thereof comprises flunarizine, or one of the pharmaceutically acceptable salts, hydrates, isomers and racemates thereof, in combination with a compound chosen from ebastine, azelastine, loperamide, astemizole, duloxetine, metixene, clemastine, thioridazine and triflupromazine, or one of the pharmaceutically acceptable salts, hydrates, isomers and racemates thereof.

In particular, according to one embodiment, the composition for use thereof comprises loperamide, or one of the pharmaceutically acceptable salts, hydrates, isomers and racemates thereof, in combination with a compound chosen from ebastine, azelastine, flunarizine, astemizole, duloxetine, metixene, clemastine, thioridazine and triflupromazine, or one of the pharmaceutically acceptable salts, hydrates, isomers and racemates thereof.

According to one embodiment, the composition for use thereof comprises astemizole, or one of the pharmaceutically acceptable salts, hydrates, isomers and racemates thereof, in combination with a compound chosen from ebastine, azelastine, flunarizine, loperamide, duloxetine, metixene, clemastine, thioridazine and triflupromazine, or one of the pharmaceutically acceptable salts, hydrates, isomers and racemates thereof.

According to one embodiment, the composition for use thereof comprises duloxetine, or one of the pharmaceutically acceptable salts, hydrates, isomers and racemates thereof, in combination with a compound chosen from ebastine, azelastine, flunarizine, loperamide, astemizole, metixene, clemastine, thioridazine and triflupromazine, or one of the pharmaceutically acceptable salts, hydrates, isomers and racemates thereof.

According to one embodiment, the composition for use thereof comprises metixene, or one of the pharmaceutically acceptable salts, hydrates, isomers and racemates thereof, in combination with a compound chosen from ebastine, azelastine, flunarizine, loperamide, astemizole, duloxetine, clemastine, thioridazine and triflupromazine, or one of the pharmaceutically acceptable salts, hydrates, isomers and racemates thereof.

According to one embodiment, the composition for use thereof comprises clemastine, or one of the pharmaceutically acceptable salts, hydrates, isomers and racemates thereof, in combination with a compound chosen from ebastine, azelastine, flunarizine, loperamide, astemizole, duloxetine, metixene, thioridazine and triflupromazine, or one of the pharmaceutically acceptable salts, hydrates, isomers and racemates thereof.

According to one embodiment, the composition for use thereof comprises thioridazine, or one of the pharmaceutically acceptable salts, hydrates, isomers and racemates thereof, in combination with a compound chosen from ebastine, azelastine, flunarizine, loperamide, astemizole, duloxetine, metixene, clemastine and triflupromazine, or one of the pharmaceutically acceptable salts, hydrates, isomers and racemates thereof.

According to one embodiment, the composition for use thereof comprises triflupromazine, or one of the pharmaceutically acceptable salts, hydrates, isomers and racemates thereof, in combination with a compound chosen from ebastine, azelastine, flunarizine, loperamide, astemizole, duloxetine, metixene, clemastine and thioridazine, or one of the pharmaceutically acceptable salts, hydrates, isomers and racemates thereof.

Preferably, the composition for use thereof comprises a combination chosen from:
ebastine and flunarizine,
ebastine and azelastine,
ebastine and loperamide,
azelastine and flunarizine,
azelastine and loperamide, and
flunarizine and loperamide,

or the pharmaceutically acceptable salts, hydrates, isomers and racemates thereof.

The compounds which may cross the blood-brain barrier, notably alimemazine (trimeprazine), atomoxetine, azelastine, biperiden, chloropyramine, citalopram, dicyclomine, diphenhydramine, duloxetine, ebastine, nefopam, orphenadrine, triflupromazine, zimelidine, are more particularly relevant for use in the treatment of neurological proteinopathies. The compounds that do not pass through the blood-brain barrier such as benzydamine, loperamide and prenylamine, are more particularly relevant for use in the treatment of non-neurological proteinopathies. It may be advantageous to combine compounds capable of passing through the blood-brain barrier with compounds which cannot do so in order to reduce the presence of prions in a more generalized manner.

It is understood that the compounds intended to be used in combination according to the present invention may be administered simultaneously, separately or sequentially.

Table 1 below gives examples of CAS identification numbers of the compounds for use according to the invention, and optionally the CAS number or the pharmaceutically acceptable salts, hydrates, isomers and racemates thereof.

TABLE 1

| Name | Examples of CAS |
|---|---|
| Amitriptyline | 50-48-6 |
| Antazoline | 91-75-8 |
| Antazoline phosphate | 154-68-7 |
| Antazoline sulfate | 24359-81-7 |
| Astemizole | 68844-77-9 |
| Atomoxetine | 83015-26-3 |
| Azelastine | 58581-89-8 |
| Benzydamine | 642-72-8 |
| Benzydamine hydrochloride | 132-69-4 |
| Biperiden | 514-65-8 |
| Cetirizine | 83881-51-0 |
| Chloropyramine | 59-32-5 |
| Chlorpromazine | 50-53-3 |
| Cinnarizine | 298-57-7 |
| Citalopram | 59729-33-8 |
| Citalopram hydrobromide | 59729-32-7 |
| Clemastine | 15686-51-8 |
| Clomipramine | 303-49-1 |
| Desipramine | 50-47-5 |
| Desloratadine | 100643-71-8 |
| Diazepam | 439-14-5 |
| Dicyclomine | 77-19-0 |
| Diphenhydramine | 58-73-1 |
| Diphenidol | 972-02-1 |
| Doxepin | 1668-19-5 |
| Duloxetine | 116539-59-4, 116817-13-1 |
| Ebastine | 90729-43-4 |
| Epinastine | 80012-43-7 |
| Ethosuximide | 77-67-8, 39122-20-8; 39122-19-5 |
| Flunarizine | 52468-60-7 |
| Fluoxetine | 54910-89-3 |
| Haloperidol | 52-86-8 |
| Imipramine | 50-49-7 |
| Isradipine | 75695-93-1 |
| Ketotifen | 34580-13-7, 34580-14-8 |
| Loperamide | 53179-11-6 |
| Metixene | 4969-02-2 |
| Metixene hydrochloride | 1553-34-0 |
| Metixene hydrochloride monohydrate | 7081-40-5 |
| Mirtazapine | 85650-52-8 |
| Nefopam | 13669-70-0 |
| Orphenadrine | 83-98-7 |
| Orphenadrine citrate | 4682-36-4 |
| Orphenadrine hydrochloride | 341-69-5 |
| Paramethadione | 115-67-3 |
| Prenylamine | 390-64-7 |
| Prenylamine lactate | 69-43-2 |
| Quinacrine (Mepacrine) | 83-89-6, 78901-94-7 |
| Quinacrine dihydrochloride | 69-05-6 |
| Quinacrine dihydrochloride dihydrate | 6151-30-0 |
| Reboxetine | 71620-89-8, 98769-84-7, |
| | 98819-76-2 |
| Thioridazine | 50-52-2 |
| Trifluoperazine | 117-89-5 |
| Triflupromazine | 146-54-3 |
| Alimemazine (Trimeprazine) | 84-96-8 |
| Zimelidine | 56775-88-3 |
| Anhydrous zimelidine hydrochloride | 60525-15-7 |
| Zonisamide | 68291-97-4 |
| Imiquimod | 99011-02-6 |
| Guanabenz | 5051-62-7 |
| 6-Aminophenanthridine | 832-68-8 |
| Tacrolimus | 104987-11-3, 109581-93-3 |
| Doxycycline | 564-25-0, 17086-28-1 |

Generally, the proteins can only exercise their function when they adopt a suitable spatial conformation referred to as a functional conformation. A bad conformation leads to numerous pathologies, in animals including humans. Examples of human proteinopathies are given below.

Prion diseases, within the meaning of the invention, encompass all diseases due to a prion in a mammal, such as for example bovine spongiform encephalopathy, Creutzfeldt-Jakob Disease (CJD), Gerstmann-Straussler-Scheinker syndrome (SGSS), fatal familial insomnia (FFI), kuru, VPSPr disease, scrapie, chronic wasting disease of cervids (CWD), feline spongiform encephalopathy, camel spongiform encephalopathy (CSE) and exotic ungulate encephalopathy.

CJD exists in several forms: the sporadic form (sCJD), the hereditary form (fCJD), the acquired or iatrogenic form (iCJD). The new variant of CJD (vCJD) is considered to be an example of the iatrogenic form of CJD. CJD is a rare transmissible encephalopathy (1 case per million individuals per year) prevalent especially between 50 and 70 years old. Death generally occurs in the year following the appearance of symptoms, which are: sleep disorders, personality changes, ataxia, aphasia, loss of vision, general weakness, muscular atrophy, myoclonus and progressive dementia.

VPSPr (variably protease sensitive prionopathy) disease is a sporadic prion disease due to the spontaneous conversion of PrP^{C} to PrP^{res} or a somatic mutation. It has similarities with CJD but the protein in PrP^{res} pathological form is less resistant to digestion by proteases. Patients present psychiatric symptoms, speech deficits (aphasia and/or dysarthria) and also cognitive deficiencies. This disease is very rare with an incidence that varies from 2 to 3 per 100 million people.

Fatal familial insomnia is a transmissible spongiform encephalopathy linked to an anomaly of the PRNP gene encoding the prion protein PrP^{C}. It affects around 40 families worldwide.

Proteins other than the prion protein PrP may be associated with proteinopathies.

For example, Lewy body disease or Lewy body dementia is a neurodegenerative disease characterized by the deposit of the alpha-synuclein protein (which makes it a synucleinopathy) within neurons. The symptoms are mainly the development of dementia, a parkinsonian syndrome, variations in cognitive performance and visual hallucinations. It is the second most common neurodegenerative dementia after Alzheimer's disease.

Parkinson's disease is also a synucleinopathy. It is associated with tremors, hypokinesia and postural instability. On a global scale, the disease is diagnosed in more than 300 000 people each year.

Alzheimer's disease is a progressive neurological disease affecting the brain and which is characterized essentially by the presence of neuritic plaques composed of aggregates of beta-amyloid peptides, resulting from the cleavage of the amyloid precursor protein (APP), and aggregates of the Tau protein. This disease is one of the most common neuropathologies and has a global incidence.

Huntington's disease or Huntington's chorea is a rare dominant genetic disease caused by the synthesis of the huntingtin protein resulting from a genetic alteration (increase in the number of Q residues encoded by exon 1 of the HTT gene) on chromosome 4. The symptoms may be motor symptoms (in particular difficulties speaking and swallowing), cognitive symptoms and psychiatric symptoms.

Amyotrophic lateral sclerosis (ALS), or Charcot disease, is characterized by a progressive degeneration of the motor neurons of the cerebral cortex with consecutive destruction of the pyramidal tract. The motricity which is affected in the disease therefore relates to both the mobility of the face (smile, speech, movement of the tongue, ability to swallow or speak) and to the mobility of the arms and legs. FUS, TDP43, SOD1, C9Orf72, KIF5A are among the proteins involved.

In type 2 diabetes, an accumulation of amyloid polypeptides (IAPP, also known as amylin) was observed in the β cells of the islets of Langerhans of the pancreases of more than 80% of patients, which would cause the destruction of the beta-pancreatic cells in diabetics. A two times higher risk of developing Alzheimer's disease was observed in patients suffering from type 2 diabetes, suggesting a cross-seeding mechanism of infectious amyloid proteins between the brain and the pancreas, which would confirm a prion-type operation in these two pathologies.

Oculopharyngeal muscular dystrophy (OPMD) is a rare dominant autosomal genetic disease which affects around 1 person per 100 000 in Europe. It is caused by an expansion of the polyalanine domain in the PABPN1 protein encoded by the PABPN1 (poly(A)-binding protein nuclear 1; 14q11.2) gene, which leads to the synthesis of a mutant protein which accumulates in the form of nuclear aggregates in the muscle cells. Thus, OPMD is likened to a proteinopathy (Harish et al. 2018).

Frontotemporal dementia is hereditary in half of cases; the most common mutations involve the 17q21-22 chromosome responsible for anomalies in the microtubule-binding tau protein, which classifies it among tauopathies. Supranuclear paralysis and corticobasal degeneration may be considered to be forms of frontotemporal dementia, since they share common neuropathological bases and similar gene mutations affecting the tau protein. The other pathogenic proteins which have been described are FUS, TDP43 and SOD1.

Preferably, the proteinopathy is chosen from Creutzfeldt-Jakob disease, Gerstmann-Straussler-Scheinker syndrome, fatal familial insomnia, kuru, VPSPr disease, Lewy body disease, Parkinson's disease, Alzheimer's disease, Huntington's disease, amyotrophic lateral sclerosis, frontotemporal dementia, type 2 diabetes, oculopharyngeal muscular dystrophy, bovine spongiform encephalopathy, scrapie, chronic wasting disease of cervids, feline spongiform encephalopathy, camel spongiform encephalopathy and exotic ungulate encephalopathy.

In particular, the proteinopathy is a human proteinopathy chosen from Creutzfeldt-Jakob disease, Gerstmann-Straussler-Scheinker syndrome, fatal familial insomnia, kuru, VPSPr disease, Lewy body disease, Parkinson's disease, Alzheimer's disease, Huntington's disease, amyotrophic lateral sclerosis, frontotemporal dementia, type 2 diabetes, oculopharyngeal muscular dystrophy.

According to an alternative, the proteinopathy is a proteinopathy affecting non-human mammals, chosen from bovine spongiform encephalopathy, scrapie, chronic wasting disease of cervids, feline spongiform encephalopathy, camel spongiform encephalopathy and exotic ungulate encephalopathy.

According to one embodiment, the invention relates to a composition comprising at least one compound chosen from ebastine, azelastine, duloxetine, atomoxetine, benzydamine, biperiden, chloropyramine, citalopram, dicyclomine, nefopam, orphenadrine, prenylamine, triflupromazine and zimelidine, for use thereof in the treatment of a proteinopathy chosen from Creutzfeldt-Jakob disease, Gerstmann-Straussler-Scheinker syndrome, fatal familial insomnia, kuru, VPSPr disease, Lewy body disease, Parkinson's disease, Alzheimer's disease, Huntington's disease, amyotrophic lateral sclerosis, frontotemporal dementia, type 2 diabetes, oculopharyngeal muscular dystrophy, bovine spongiform encephalopathy, scrapie, chronic wasting disease of cervids, feline spongiform encephalopathy, camel spongiform encephalopathy and exotic ungulate encephalopathy.

Preferably, the invention relates to a composition comprising ebastine for use thereof in the treatment of a proteinopathy chosen from Creutzfeldt-Jakob disease, Gerstmann-Straussler-Scheinker syndrome, fatal familial insomnia, kuru, VPSPr disease, Lewy body disease, Parkinson's disease, Alzheimer's disease, Huntington's disease, amyotrophic lateral sclerosis, frontotemporal dementia, type 2 diabetes, oculopharyngeal muscular dystrophy, bovine spongiform encephalopathy, scrapie, chronic wasting disease of cervids, feline spongiform encephalopathy, camel spongiform encephalopathy and exotic ungulate encephalopathy.

According to one alternative, the invention relates to a composition comprising azelastine for use thereof in the treatment of a proteinopathy chosen from Creutzfeldt-Jakob disease, Gerstmann-Straussler-Scheinker syndrome, fatal familial insomnia, kuru, VPSPr disease, Lewy body disease, Parkinson's disease, Alzheimer's disease, Huntington's disease, amyotrophic lateral sclerosis, frontotemporal dementia, type 2 diabetes, oculopharyngeal muscular dystrophy, bovine spongiform encephalopathy, scrapie, chronic wasting disease of cervids, feline spongiform encephalopathy, camel spongiform encephalopathy and exotic ungulate encephalopathy.

According to another alternative, the invention relates to a composition comprising duloxetine for use thereof in the treatment of a proteinopathy chosen from Creutzfeldt-Jakob disease, Gerstmann-Straussler-Scheinker syndrome, fatal familial insomnia, kuru, VPSPr disease, Lewy body disease, Parkinson's disease, Alzheimer's disease, Huntington's disease, amyotrophic lateral sclerosis, frontotemporal dementia, type 2 diabetes, oculopharyngeal muscular dystrophy, bovine spongiform encephalopathy, scrapie, chronic wasting disease of cervids, feline spongiform encephalopathy, camel spongiform encephalopathy and exotic ungulate encephalopathy.

According to another alternative, the invention relates to a composition comprising triflupromazine for use thereof in the treatment of a proteinopathy chosen from Creutzfeldt-Jakob disease, Gerstmann-Straussler-Scheinker syndrome, fatal familial insomnia, kuru, VPSPr disease, Lewy body disease, Parkinson's disease, Alzheimer's disease, Huntington's disease, amyotrophic lateral sclerosis, frontotemporal dementia, type 2 diabetes, oculopharyngeal muscular dystrophy, bovine spongiform encephalopathy, scrapie, chronic wasting disease of cervids, feline spongiform encephalopathy, camel spongiform encephalopathy and exotic ungulate encephalopathy.

According to another embodiment, the invention relates to a composition comprising at least one compound chosen from ebastine, azelastine, duloxetine, atomoxetine, benzydamine, biperiden, chloropyramine, citalopram, dicyclomine, nefopam, orphenadrine, prenylamine, triflupromazine and zimelidine, preferably from ebastine, azelastine, duloxetine and triflupromazine, for use thereof in the treatment of a proteinopathy linked to the prion protein PrP, preferably a human proteinopathy, preferably chosen from Creutzfeldt-Jakob disease, Gerstmann-Straussler-Scheinker syndrome, fatal familial insomnia, kuru and VPSPr disease.

The compositions for the treatment of proteinopathies according to the invention may be administered in combination with the treatments currently in development for this indication, in particular an immunotherapy.

Administration

The compounds according to the invention are known to those skilled in the art for their use in other therapeutic applications. The known dosage and posology may of course be adapted within the context of the present invention by the practitioner according to their general knowledge. Examples of trade names and of doses that can be administered are given below, for illustrative purposes only:
ebastine (trade name e.g. “Kestin”): from about 1 to about 30 mg per day, preferably from about 10 to about 20 mg per day, in one, two or three doses.
azelastine (trade name e.g. “Allergodil”): from about 0.30 to about 2.00 mg per day, preferably between about 0.50 and about 1.00 mg per day, notably 0.56 mg per day, in one, two or three doses.
duloxetine (trade name e.g. “Cymbalta”, “Yentreve”, “Xeristar” or “AriClaim”): from about 15 to about 150 mg per day, preferably about 30, about 60, about 90 or about 120 mg per day, in one, two or three doses, more preferably about 60 mg per day in a single dose.
atomoxetine (trade name e.g. “Strattera”) in the form of tablets at the dose of from about 0.5 mg/kg/day to about 1.8 mg/kg/day, preferably about 1.2 mg/kg/day.
benzydamine notably in the form of benzydamine hydrochloride (trade name e.g. “Opalgyne”), for example in pastilles at the dose of about 9 mg/day, or in solution at the dose of about 300 mg/day, preferably of about 200 mg/day.
biperiden (trade name e.g. “Akineton”) in the form of tablets at the dose of from about 1 mg/day to about 12 mg/day, preferably about 4 mg/day.
chloropyramine (trade name e.g. “Suprastin”) in the form of tablets at the dose of from about 75 mg/day to about 150 mg/day, preferably about 100 mg/day.
citalopram, notably in the form of citalopram hydrobromide (trade name e.g. “Celapram”, “Celexa”, “Cipram”, “Cipramil”, “Ecosol”, Mepha, “Recital”, “Seropram” etc.) in the form of tablets at the dose of from about 20 mg/day to about 40 mg/day, preferably about 20 mg/day.
dicyclomine (trade name e.g. “Bentyl”, “Dibent”, “Dicyclocot”) in the form of tablets at the dose of from about 4 mg/day to about 40 mg/day, preferably about 4 mg/day.
nefopam (trade name e.g. “Acupan”) in the form of an injectable solution at the dose of from about 20 mg/day to about 120 mg/day, preferably from about 20 to about 80 mg/day.
orphenadrine (trade name e.g. “Norflex”), notably orphenadrine citrate or orphenadrine hydrochloride, in the form of an injectable solution or of tablets, at the dose of from about 60 mg/day to about 120 mg/day, preferably about 60 mg/day.
prenylamine (trade name e.g. “Segontin”), notably prenylamine lactate, in the form of tablets at the dose of from about 60 mg/day to about 180 mg/day, preferably about 60 mg/day.
triflupromazine (trade name e.g. “Vesprin”) in the form of tablets at the dose of from about 2 mg/day to about 40 mg/day, preferably about 10 mg/day.
zimelidine (trade name e.g. “Normud” or “Zelmid”) in the form of tablets at the dose of from about 10 to about 200 mg per day, preferably from about 25 to about 100 mg per day, preferably about 50 mg per day, in one or two doses.

Compositions as such

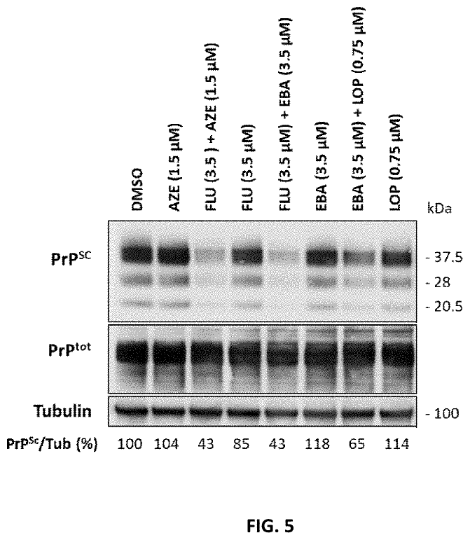

The invention also relates to compositions as such, comprising a combination of compounds as described previously. Specifically, the inventors have shown that such combinations could have a synergistic effect on the improvement of the anti-prion activity, in particular in models of cell cultures (FIG. 5). Specifically, the reduction in the degree of accumulation of PrP^{Sc} obtained by means of combinations of compounds is markedly greater than the sum of the effects obtained by the use of the compounds alone. At 1.5 μM and 3.5 μM, azelastine and flunarizine do not change the degree of PrP^{Sc} (104% and 85%, respectively). However, the simultaneous treatment of the cells with 1.5 μM of azelastine and 3.5 μM of flunarizine reduces the degree of PrP^{Sc} to 43% which is markedly greater than the addition of the effects of the two individual molecules (15%). Azelastine used alone at 1.5 μM also does not have an effect on the degree of PrP^{Sc} (104%), whereas its combination with flunarizine at 3.5 μM (which alone resulted in a degree of PrP^{Sc} of 85%) makes it possible to reduce this number to 43%. Loperamide used alone at 0.75 μM has no effect on the degree of PrP^{Sc} whereas its combination with ebastine at 3.5 μM lowers the degree of PrP^{Sc} to 65%.

Thus, the invention relates to a composition comprising at least one compound chosen from ebastine, azelastine, duloxetine, atomoxetine, benzydamine, biperiden, chloropyramine, citalopram, dicyclomine, nefopam, orphenadrine, prenylamine, triflupromazine and zimelidine, or one of the pharmaceutically acceptable salts, hydrates, isomers and racemates thereof, said compound being in combination with at least one different compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof.

For example, according to one embodiment, the composition comprises ebastine, or one of the pharmaceutically acceptable salts, hydrates, isomers and racemates thereof, in combination with at least one compound chosen from flunarizine, loperamide, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof.

According to one variant, the composition comprises azelastine, or one of the pharmaceutically acceptable salts, hydrates, isomers and racemates thereof, in combination with at least one compound chosen from flunarizine, loperamide, ebastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof.

According to another variant, the composition comprises duloxetine, or one of the pharmaceutically acceptable salts, hydrates, isomers and racemates thereof, in combination with at least one compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof.

According to one embodiment, the composition comprises at least two compounds chosen from ebastine, azelastine, duloxetine, atomoxetine, benzydamine, biperiden, chloropyramine, citalopram, dicyclomine, nefopam, orphenadrine, prenylamine, triflupromazine and zimelidine, or the pharmaceutically acceptable salts, hydrates, isomers and racemates thereof.

According to one embodiment, the composition comprises at least two compounds chosen from ebastine, azelastine, flunarizine, loperamide, astemizole, duloxetine, metixene, clemastine, thioridazine and triflupromazine, preferably from ebastine, flunarizine, loperamide and azelastine, or the pharmaceutically acceptable salts, hydrates, isomers and racemates thereof.

According to one embodiment, the composition comprises ebastine, or one of the pharmaceutically acceptable salts, hydrates, isomers and racemates thereof, in combination with a compound chosen from azelastine, flunarizine, loperamide, astemizole, duloxetine, metixene, clemastine, thioridazine and triflupromazine, or one of the pharmaceutically acceptable salts, hydrates, isomers and racemates thereof.

According to one embodiment, the composition comprises flunarizine, or one of the pharmaceutically acceptable salts, hydrates, isomers and racemates thereof, in combination with a compound chosen from ebastine, azelastine, loperamide, astemizole, duloxetine, metixene, clemastine, thioridazine and triflupromazine, or one of the pharmaceutically acceptable salts, hydrates, isomers and racemates thereof.

In particular, according to one embodiment, the composition comprises loperamide, or one of the pharmaceutically acceptable salts, hydrates, isomers and racemates thereof, in combination with a compound chosen from ebastine, azelastine, flunarizine, astemizole, duloxetine, metixene, clemastine, thioridazine and triflupromazine, or one of the pharmaceutically acceptable salts, hydrates, isomers and racemates thereof.

According to one embodiment, the composition comprises azelastine, or one of the pharmaceutically acceptable salts, hydrates, isomers and racemates thereof, in combination with a compound chosen from ebastine, flunarizine, loperamide, astemizole, duloxetine, metixene, clemastine, thioridazine and triflupromazine, or one of the pharmaceutically acceptable salts, hydrates, isomers and racemates thereof.

According to one embodiment, the composition comprises astemizole, or one of the pharmaceutically acceptable salts, hydrates, isomers and racemates thereof, in combination with a compound chosen from ebastine, azelastine, flunarizine, loperamide, duloxetine, metixene, clemastine, thioridazine and triflupromazine, or one of the pharmaceutically acceptable salts, hydrates, isomers and racemates thereof.

According to one embodiment, the composition comprises metixene, or one of the pharmaceutically acceptable salts, hydrates, isomers and racemates thereof, in combination with a compound chosen from ebastine, azelastine, flunarizine, loperamide, astemizole, duloxetine, clemastine, thioridazine and triflupromazine, or one of the pharmaceutically acceptable salts, hydrates, isomers and racemates thereof.

According to one embodiment, the composition comprises duloxetine, or one of the pharmaceutically acceptable salts, hydrates, isomers and racemates thereof, in combination with a compound chosen from ebastine, azelastine, flunarizine, loperamide, astemizole, metixene, clemastine, thioridazine and triflupromazine, or one of the pharmaceutically acceptable salts, hydrates, isomers and racemates thereof.

According to one embodiment, the composition comprises clemastine, or one of the pharmaceutically acceptable salts, hydrates, isomers and racemates thereof, in combination with a compound chosen from ebastine, azelastine, flunarizine, loperamide, astemizole, duloxetine, metixene, thioridazine and triflupromazine, or one of the pharmaceutically acceptable salts, hydrates, isomers and racemates thereof.

According to one embodiment, the composition comprises thioridazine, or one of the pharmaceutically acceptable salts, hydrates, isomers and racemates thereof, in combination with a compound chosen from ebastine, azelastine, flunarizine, loperamide, astemizole, duloxetine, metixene, clemastine and triflupromazine, or one of the pharmaceutically acceptable salts, hydrates, isomers and racemates thereof.

According to one embodiment, the composition comprises triflupromazine, or one of the pharmaceutically acceptable salts, hydrates, isomers and racemates thereof, in combination with a compound chosen from ebastine, azelastine, flunarizine, loperamide, astemizole, duloxetine, metixene, clemastine and thioridazine, or one of the pharmaceutically acceptable salts, hydrates, isomers and racemates thereof.

Preferably, the composition comprises a combination from among:
ebastine and flunarizine,
ebastine and azelastine,
ebastine and loperamide,
azelastine and flunarizine,
azelastine and loperamide, and
flunarizine and loperamide,

or the pharmaceutically acceptable salts, hydrates, isomers and racemates thereof.

The compositions according to the invention typically comprise one or more acceptable excipients or carriers. Thus, the compositions according to the invention as such or for use thereof in the treatment of a proteinopathy are generally mixed with pharmaceutically acceptable carriers or excipients.

The term “pharmaceutically acceptable carrier or excipient” denotes here a compound of pharmaceutical grade which improves the administration, stability or bioavailability of a compound and can be metabolized by a subject to whom it is administered and is not toxic for said subject. The preferred excipients according to the invention comprise any one of the excipients commonly used in pharmaceutical products, such as for example microcrystalline cellulose, lactose, starch and soy powder.

Thus, another subject according to the invention relates to a process for manufacturing pharmaceutical compositions comprising the mixture of the compounds or combinations described above with at least one or more pharmaceutically acceptable carriers or excipients.

Use of PFAR Inhibitors

One subject according to the invention consists of the use of compounds as novel PFAR inhibitors. To better understand the role of PFAR in physiopathological mechanisms, the inventors have discovered that drugs known for their activity relative to other cellular targets were also effective in the inhibition of PFAR.

The use of these compounds to inhibit PFAR is particularly useful in the understanding of the role of ribosome and the etiology of protein misfolding diseases.

The inventors have thus surprisingly discovered that alimemazine (trimeprazine), amitriptyline, astemizole, atomoxetine, azelastine, benzydamine, biperiden, chloropyramine, citalopram, clemastine, clomipramine, desipramine, desloratadine, dicyclomine, diphenhydramine, doxepin, duloxetine, ebastine, fluoxetine, haloperidol, imipramine, loperamide, nefopam, orphenadrine, prenylamine, reboxetine, thioridazine, trifluoperazine, triflupromazine, chlorpromazine, quinacrine (mepacrine) and zimelidine could be used as candidates for inhibiting PFAR activity.

According to a preferred embodiment, the invention relates to the use, preferably in vitro or ex vivo, of at least one compound chosen from alimemazine (trimeprazine), amitriptyline, astemizole, atomoxetine, azelastine, benzydamine, biperiden, chloropyramine, citalopram, clemastine, clomipramine, desipramine, desloratadine, dicyclomine, diphenhydramine, doxepin, duloxetine, ebastine, fluoxetine, haloperidol, imipramine, loperamide, nefopam, orphenadrine, prenylamine, reboxetine, thioridazine, trifluoperazine, triflupromazine, chlorpromazine, quinacrine (mepacrine) and zimelidine, preferably at least one compound chosen from ebastine, azelastine, loperamide, duloxetine and triflupromazine, or one of the pharmaceutically acceptable salts, hydrates, isomers and racemates thereof, to inhibit PFAR. The combinations of these compounds are also envisaged.

The use of these compounds to inhibit PFAR may be combined with the use, preferably in vitro or ex vivo, of at least one different compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof.

For example, according to one particular embodiment, the invention relates to the use, preferably in vitro or ex vivo, of a composition comprising ebastine, or one of the pharmaceutically acceptable salts, hydrates, isomers and racemates thereof, in combination with at least one compound chosen from flunarizine, loperamide, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof, to inhibit PFAR.

According to another particular embodiment, the invention relates to the use, preferably in vitro or ex vivo, of a composition comprising azelastine, or one of the pharmaceutically acceptable salts, hydrates, isomers and racemates thereof, in combination with at least one compound chosen from flunarizine, loperamide, ebastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof, to inhibit PFAR.

According to another particular embodiment, the invention relates to the use, preferably in vitro or ex vivo, of a composition comprising duloxetine, or one of the pharmaceutically acceptable salts, hydrates, isomers and racemates thereof, in combination with at least one compound chosen from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof, to inhibit PFAR.

According to another particular embodiment, the invention relates to the use, preferably in vitro or ex vivo, of a composition comprising loperamide, or one of the pharmaceutically acceptable salts, hydrates, isomers and racemates thereof, in combination with at least one compound chosen from flunarizine, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof, to inhibit PFAR.

During the use of the compounds present in combination to inhibit PFAR, the compounds may be used simultaneously, separately or sequentially.

Preferably, the uses of the compounds according to the invention are in vitro and/or ex vivo uses.

According to one particular embodiment, the use of the compounds to inhibit the PFAR activity is an in vitro use on cells, such as isolated or clustered, eukaryotic or prokaryotic, animal or plant cells, for example in organoids. Particularly, the use of the compounds to inhibit the PFAR activity is carried out in yeasts.

According to another embodiment, the use of the compounds to inhibit the PFAR activity is an ex vivo use on organs or fragments of organs isolated from an animal or from a plant and which are not intended to be reintroduced into the animals or the plants. Particularly, the use of compounds to inhibit the PFAR activity is carried out in slices of mouse cerebella.

### EXAMPLES

The invention will be better understood by means of the examples given below by way of illustration and without limitation.

Screening of Molecule Banks

The inventors have opted for the SOSA (Selective optimization of side activities) approach which makes it possible to carry out therapeutic repositioning. This approach proposes to screen preferably drugs already in clinical practice or in the course of clinical trials in order to determine if they are likely to have additional pharmacological targets (Wermuth 2004). Given that their toxicity, their safety and their bioavailability in humans have already been evaluated, the drugs derived from chemical libraries based on the SOSA approach could rapidly be administered as compassionate treatment to people suffering from prion diseases for which no treatment is currently available. Within this context, the Prestwick chemical library was screened, enabling the identification of flunarizine, possessing an anti-prion activity and which is a PFAR inhibitor (Nguyen 2013).

The latest version of DrugBank (version 5.1.0 dated Feb. 4, 2018) was downloaded directly from the website https://www.drugbank.ca and filtered so as to retain the FDA-approved compounds and abandoned compounds, i.e. 1887 compounds. These compounds were retrieved as multiconformer files in the oeb.gz format using OMEGA v2.5.1.4. (OpenEye Scientific Software, http://www.eyesopen.com/).

An ROCS (“Rapid Overlay of Chemical Structures”) search makes it possible to identify compounds by similarity in the superposition of the shapes and/or pharmacophores, using only the heavy atoms of a ligand and ignoring the hydrogens. The degree of shape and/or pharmacophore similarity is illustrated by a “ComboScore” score. This ROCS search was carried out on the basis of flunarizine. Flunarizine has a pharmacophore structure composed of three aromatic groups, and of two nitrogen atoms, which may interact with the target by means of interactions with electrostatics or hydrogen bond acceptors. The experimentally crystallized structure of flunarizine was obtained from The Cambridge Crystallographic Data Centre (www.ccdc.cam.ac.uk/structures, compound identifier: “JOBSIE”).

Among the 1887 compounds selected by this method, 45 compounds were chosen: amitriptyline, antazoline, astemizole, atomoxetine, azelastine, benzydamine, biperiden, cetirizine, chloropyramine, chlorpromazine, cinnarizine, citalopram, clemastine, clomipramine, desipramine, desloratadine, diazepam, dicyclomine, diphenhydramine, diphenidol, doxepin, duloxetine, ebastine, epinastine, ethosuximide, fluoxetine, haloperidol, imipramine, isradipine, ketotifen, loperamide, metixene, mirtazapine, nefopam, orphenadrine, paramethadione, prenylamine, quinacrine (mepacrine), reboxetine, thioridazine, trifluoperazine, triflupromazine, alimemazine (trimeprazine), zimelidine and zonisamide. Table 2 below gives the ComboScore obtained for these compounds, with the exception of ebastine for which the value was unable to be calculated (“nd” in the table).

TABLE 2

| Compounds | ComboScore |
|---|---|
| amitriptyline | 113 |
| antazoline | 152 |
| astemizole | 36 |
| atomoxetine | 41 |
| azelastine | 165 |
| benzydamine | 476 |
| biperiden | 33 |
| cetirizine | 10 |
| chloropyramine | 157 |
| chlorpromazine | 191 |
| cinnarizine | 2 |
| citalopram | 23 |
| clemastine | 27 |
| clomipramine | 153 |
| desipramine | 161 |
| desloratadine | 100 |
| diazepam | 747 |
| dicyclomine | 134 |
| diphenhydramine | 21 |
| diphenidol | 24 |
| doxepin | 149 |
| duloxetine | 84 |
| ebastine | 14 |
| epinastine | 850 |
| ethosuximide | 1839 |
| flunarizine | 1 |
| fluoxetine | 38 |
| haloperidol | 136 |
| imipramine | 122 |
| isradipine | 422 |
| ketotifen | 95 |
| loperamide | 25 |
| metixene | 121 |
| mirtazapine | 287 |
| nefopam | 592 |
| orphenadrine | 15 |
| paramethadione | 1845 |
| prenylamine | 6 |
| quinacrine (mepacrine) | 655 |
| reboxetine | 85 |
| thioridazine | 188 |
| trifluoperazine | 202 |
| triflupromazine | 325 |
| alimemazine (trimeprazine) | 248 |
| zimelidine | 175 |
| zonisamide | 1528 |

The compounds were tested for their activity against the prion PrP^{Sc} in cell culture, as described below.

The compounds According to the Invention are Effective for Inhibiting PFAR

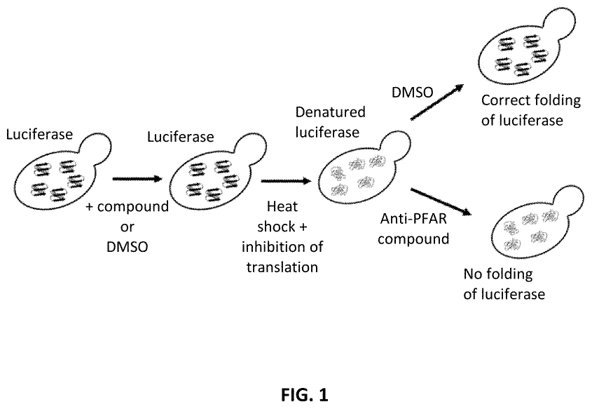

The experimental protocol for identifying the compounds capable of inhibiting PFAR activity in vivo was covered by Blondel et al. (2016) and is depicted in FIG. 1. In detail, the [psi-] Itv1Δ/hsp104Δ 74-D694 strain (ΔLΔH, Blonde) et al. 2016), deleted of the HSP104 gene and enriched in PFAR owing to the deletion of the LTV1 gene, was converted by the pDCM90 plasmid (Hasin et al. 2014; Parsell et al. 1994) enabling the constituent expression of the LuxAB (temperature-sensitive bacterial luciferase) polypeptide. The converted yeasts were exponentially cultured at 29° C. The cells were treated with the indicated concentrations of compounds or of DMSO for 2 hours before the heat shock. The LuxAB was then heat-inactivated during an incubation at 43.5° C. for 60 minutes. To prevent the synthesis of fresh luciferase, 10 μg/ml of cycloheximide (Sigma Aldrich) were added after 45 minutes at 43.5° C. What is measured is therefore the re-establishment of the functional conformation overtime, by the PFAR of the yeast ribosomes, of the LuxAB enzymes present at the time of the heat shock. The activity of the LuxAB was measured 60 minutes after the heat shock (corresponding to the time 0 minute in FIG. 1), and the cells were placed under culture conditions at 29° C. for the periods indicated, the luciferase activity being measured at intervals of 30 minutes by the addition of 10 μl of n-decylaldehyde (Decanal, Sigma Aldrich) to 120 μl of yeast culture. Luminescence was measured using a Varioscan microplate reader (ThermoFisher). Luciferase activity was then expressed as a percentage of the activity measured before the heat shock treatment for each strain.

Chlorpromazine, quinacrine, azelastine, thioridazine, triflupromazine and guanabenz chloride were acquired from Sigma Aldrich (United States); astemizole, duloxetine, clemastine and ebastine were acquired from CarboSynth (United Kingdom); metixene was purchased from LGC (Laboratoire de Genie Chimique in Toulouse, France).

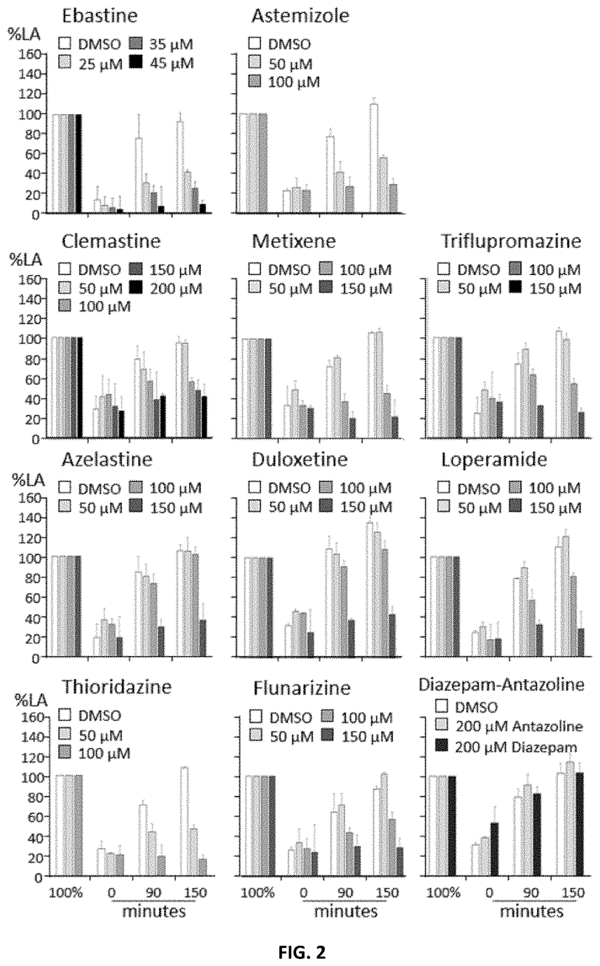

As shown in FIG. 2, for DMSO (all of the panels), the heat shock reduced the LuxAB activity to about 30% of its initial activity. In the presence of DMSO, the LuxAB activity increased with time, which indicates that it regained its active conformation.

The compounds according to the invention inhibit PFAR in vivo in yeast. Imiquimod, guanabenz and 6AP were used as positive controls (Oumata et al. 2013; Tribouillard-Tanvier et al. 2008a). At concentrations of 150 μM and 200 μM of imiquimod, the LuxAB activity does not manage to regain its initial level.

FIG. 2 shows that all of the compounds tested inhibit the PFAR activity: from 25 μM for ebastine, 50 μM for astemizole and thioridazine, 100 μM for clemastine, metixene, flunarizine and triflupromazine, and from 150 μM for azelastine, duloxetine and loperamide.

The Compositions According to the Invention are Effective as Anti-Prions In Vitro

The in vitro experiments were carried out as described in the literature (Nguyen et al. 2014a; Oumata et al. 2013; Archer et al. 2004). MovS6 cells chronically infected with ovine 127S prion strain were treated for 6 days with the indicated concentrations of compounds. They were then lysed, and 250 μg of cell lysates were digested by proteinase K. The detection of PrP^{tot} was performed on 25 μg of crude cell lysate. The PrP proteins were then immunostained using an anti-PrP antibody (Sha31, Bertin Pharma). It was thus possible to define the half maximal inhibitory concentration IC₅₀.

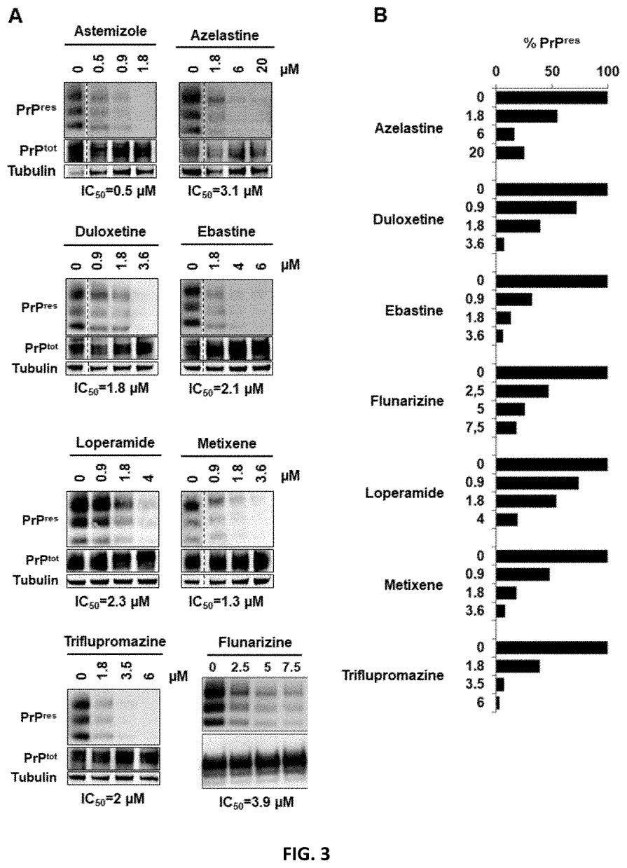

Table 3 gives the details of the doses tested and the IC₅₀ values obtained, except for chlorpromazine and quinacrine for which the IC₅₀ values are from the literature. The western blots are presented in FIG. 3.

The compounds that are most effective in vitro are those for which the IC₅₀ is minimal. It emerges from Tables 2 and 3 that the anti-PrP^{Sc} activity is not correlated to the similarity of the compound to flunarizine (the lower the ComboScore, the closer the compound is to flunarizine).

TABLE 3

| Compounds | Doses tested (μM) | IC₅₀ (μM) |
|---|---|---|
| amitriptyline | 0; 1.8; 6; 20 | 10 |
| astemizole | 0; 0.5; 0.9; 1.8 | 0.5 |
| atomoxetine | 0; 1.8; 6; 20 | 3.9 |
| azelastine | 0; 1.8; 6; 20 | 3.1 |
| benzydamine | 0; 1.8; 6; 20 | 2.7 |
| biperiden | 0; 1.8; 6; 20 | 9.2 |
| chloropyramine | 0; 1.8; 3.5; 6 | 18.5 |
| chlorpromazine | From the literature | 3 |
| citalopram | 0; 1.8; 6; 20 | 10.4 |
| clemastine | 0; 1.8; 3.5; 6 | 2 |
| clomipramine | 0; 1.8; 6; 20 | 11 |
| desipramine | 0; 1.8; 6; 20 | 9.5 |
| desloratadine | 0; 1.8; 6; 20 | 2.9 |
| dicyclomine | 0; 1.8; 6; 20 | 3.6 |
| diphenhydramine | 0; 1.8; 6; 20 | 14.8 |
| doxepin | 0; 1.8; 6; 20 | 12.7 |
| duloxetine | 0; 0.9; 1.8; 3.6 | 1.8 |
| ebastine | 0; 1.8; 4; 6 | 2.1 |
| flunarizine | 0; 2.5; 5; 7.5; 10; 12.5; 15 | 3.9 |
| fluoxetine | 0; 1.8; 4; 6 | 6.8 |
| haloperidol | 0; 1.8; 6; 20 | 12.3 |
| imipramine | 0; 1.8; 6; 20 | 11 |
| loperamide | 0; 0.9; 1.8; 4 | 2.3 |
| metixene | 0; 0.9; 1.8; 3.6 | 1.3 |
| nefopam | 0; 1.8; 6; 20 | Negative |
| orphenadrine | 0; 1.8; 6; 20 | 9.1 |
| prenylamine | 0; 1.8; 4; 6 | 3.8 |
| quinacrine (mepacrine) | From the literature | 0.3 |
| reboxetine | 0; 1.8; 6; 20 | 10.6 |
| thioridazine | 0; 1.8; 3.5; 6 | 1.7 |
| trifluoperazine | 0; 1.8; 3.5; 6 | 3.4 |
| triflupromazine | 0; 1.8; 3.5; 6 | 2 |
| alimemazine (trimeprazine) | 0; 1.8; 6; 20 | 11.4 |
| zimelidine | 0; 1.8; 6; 20 | 14.3 |

The Compositions According to the Invention are Effective as Anti-Prions in an Organotypic Model

The experiments on animals were carried out in strict compliance with European directives EU 2010/63 and were approved by the French Minister of Higher Education, Research and

Innovation (GMO authorization no. 4292). Every effort was taken to minimize the suffering of the animals.

The antiprion activity of the compounds against the prion PrP^{Sc} was evaluated in an organotypic test in which slices of mouse cerebella were infected by the prion (strain 127S) in culture, as described above (Nguyen et al. 2014a and b; Falsig et al. 2008) using tg338 transgenic mice overexpressing the VRQ allele of the ovine prion protein (Vilotte et al. 2001). The preparation and culturing of slices were carried out as described elsewhere (Nguyen et al. 2014a and b; Falsig et al. 2008) except for infection by the prion which was carried out the day the cerebella were cut, for 1 hour in ice with 2 μg/ml of a brain homogenate prepared from tg338 mice at the terminal stage of the disease experimentally infected with the prion 127S strain. Seven days after infection, the compounds were added to the cerebellar slices at a concentration of from 20 to 30 μM. The control was performed by adding only DMSO (0.7%). The cerebellar slices were then cultured for an additional 21 days before the samplings. Fresh compounds were added at each change of culture medium (3 times per week) at the concentrations indicated.

The cerebellar slices (at least 7 slices per condition) were lysed and homogenized in 350 μL of lysis buffer (0.5% sodium deoxycholate, 0.5% Triton-X100, 5 mM Tris-HCl pH 7.5) by means of a homogenizer (Beadbug, Benchmark Scientific, United States) in the presence of glass beads (reference #079053, Dutscher, France) as described in the literature (Nguyen et al. 2014a and b; Falsig et al. 2008).

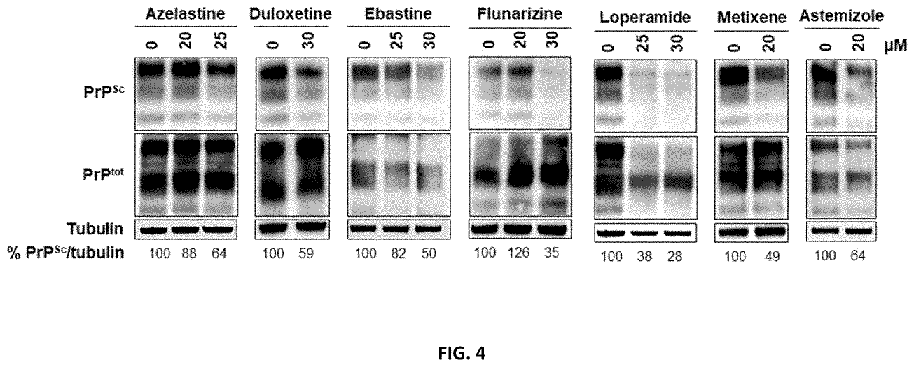

Astemizole, azelastine, duloxetine, ebastine, flunarizine, loperamide and metixene were tested according to this experimental protocol and gave good ex vivo anti-PrP^{Sc} activity results (FIG. 4).

Combinations and Synergy

The compounds were then tested in combinations.

MovS6 cells chronically infected with strain 127S of ovine (scrapie) prion PrP^{Sc} were treated with the compounds alone or in combination for 6 days. The concentrations of compounds used were 3.5 μM for flunarizine, 1.5 μM for azelastine, 1.5 μM for ebastine and 0.75 μM for loperamide. DMSO was used as control (0.7%). The cell lysates were then subjected to digestion by proteinase K to specifically reveal the PrP^{Sc} prion proteins by immunoblot. The effects of flunarizine on the steady-state level of PrP (PrP^{tot}) were determined on the same treated MovS6 cell lysates in the absence of proteinase K treatment. After cell lysis, the samples were digested by proteinase K in order to reveal the content of PrP^{Sc} by immunoblot. The compounds in combinations are more effective than the compounds alone.

FIG. 5 shows that the combinations of compounds have a markedly greater effect on the degree of accumulation of PrP^{Sc} than the addition of the effects of the compounds alone. This surprising result demonstrates the existence of a synergy between the compounds.

All these results show that the compounds reduce the PrP^{Sc} load and demonstrate their therapeutic potential.

The Compositions According to the Invention are Effective in the Treatment of Oculopharyngeal Muscular Dystrophy (OPMD) In Vitro

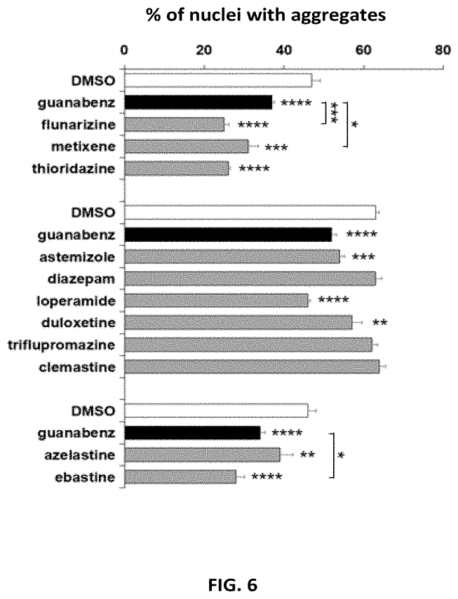

Murine myoblasts H2K D7e (Raz et al. 2011) were seeded to 80% confluency in 8-well plates (lbidi) the wells of which were covered with a layer of matrigel ( 1/10th) and contained proliferation medium composed of DMEM, heat-inactivated fetal bovine serum, penicillin, streptomycin, geneticin, chicken embryo extract and interferon-gamma. 24 hours after seeding, the cells were differentiated for 4 days in a differentiation medium composed of DMEM, horse serum (10%) and a penicillin/streptomycin mixture (1%). After 2 days of differentiation, half of the medium is replaced by fresh medium containing the test compound dissolved in DMSO to attain a final concentration of 10 μM in the well (3 wells per condition). The control well contains DMSO diluted to 1/100th. After 4 days of differentiation, the myotubes were fixed with 4% paraformaldehyde for 15 minutes then permeabilized with 0.1% Triton X-100 (CAS no. 9002-93-1). PABPN1 was stained by immunofluorescence (antibody ab75855, Abcam, 1/200) and F actin was stained by Phalloidin coupled to Alexa fluor 555 ( 1/400). Photographs were taken at 40× magnification. Between 500 and 700 nuclei were counted per well. Guanabenz was used as positive control. The results are presented in FIG. 6 and demonstrate that flunarizine, metixene, thioridazine, astemizole, loperamide, duloxetine, azelastine and ebastine reduce the amount of muscle cell nuclei containing PABPN1 aggregates. Flunarizine, metixene and ebastine are more active than guanabenz. Triflupromazine and clemastine are negative, like the negative control used (diazepam).

The Compositions According to the Invention are Effective in the Treatment of Oculopharyngeal Muscular Dystrophy (OPMD) In Vivo in a Drosophila Model

The model used is a fruit fly model for OPMD in which the mammalian protein PABPN1 with a repetition of 17 alanines (PABPN1-17a1a) is specifically expressed in the muscles of Drosophila (Chartier et al. 2006; Chartier et al. 2015). These models reproduce the characteristics of the disease, i.e. weakness and progressive muscular degeneration, and also the formation of nuclear PABPN1 aggregates. Thus, the expression of PABPN1-17ala in the indirect wing muscles leads to an abnormal wing posture, resulting from the disorder of the muscle function and degeneration of the muscles (Chartier et al. 2006).

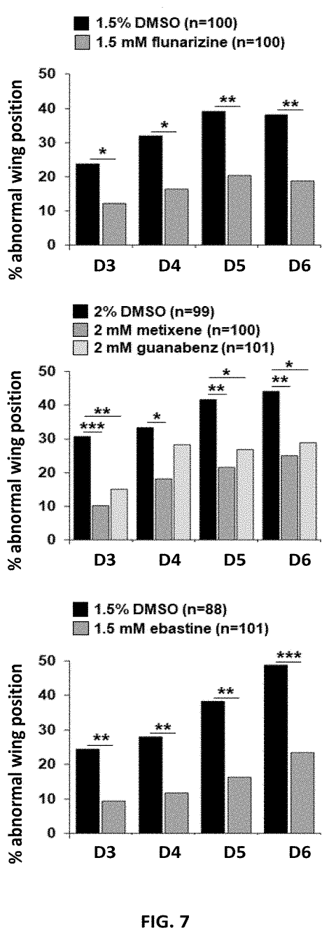

Male OPMD fruit flies (Act-88F-PABPN1-17ala/+) resulting from the breeding (Females w¹¹⁸; Act-88F-PABPN1-17ala/TM3, Sb×Males w¹¹¹⁸) were brought into contact with a nutrient agar medium containing either 1.5% of DMSO or 1.5 mM of flunarizine, or 2% of DMSO, 2 mM of metixene or 2 mM of guanabenz (positive control), or 1.5% of DMSO or 1.5 mM of ebastine. Guanabenz was used as positive control since its activity has already been described for OPMD (Barbezier et al. 2011; Malerba et al. 2019). The nutrient medium containing the drug compounds was changed every day for 6 days. FIG. 7 shows that the treatment with flunarizine (top panel) and with metixene (middle panel) and ebastine (bottom panel) makes it possible to reduce the number of fruit flies having an abnormal phenotype of the wing position.

The compounds were administered in the food, prepared as follows: an instant Drosophila medium (Carolina Biological Supply Company) was reconstituted in each tube with a 1% solution of yeast in water, supplemented by the compounds dissolved in DMSO with an increasing concentration, or with DMSO alone. Each tube contained 2 ml of reconstituted medium. The compounds were administered orally in fresh food offered to adult flies every day from day 2 to day 5. Guanabenz, the activity of which has already been described for OPMD (Barbezier et al. 2011; Malerba et al. 2019), was used as control at a concentration of 2 mM. Flunarizine was administered at 1.5 mM. Metixene was administered at a concentration of 2 mM. Ebastine was tested at 1.5 mM. The effect of the compounds was quantified by the daily measurement of the number of flies presenting an abnormal wing posture, corresponding to the flies expressing PABPN1-17ala, from day 3 to day 6 of the adult age: groups of five males per condition are placed in an empty tube and the number of males having an abnormal wing position is recorded by direct observation of the flies in the tube, without anesthesia. The total number of flies analysed for each condition is indicated in FIG. 7.

The compounds according to the invention are effective in an in vivo model of OPMD. Flunarizine, metixene and ebastine displayed beneficial effects by enabling the significant reduction (chi-squared statistical test) of the number of flies presenting an abnormal wing posture relative to the control flies (administration of DMSO alone) (FIG. 7).

Together, these results confirm that the compounds identified by the inventors are good candidates for use in inhibiting PFAR, but also for their use, alone or in combinations, in the treatment of proteinopathies, for example prion diseases.

### REFERENCES

Barbezier, N., Chartier, A., Bidet, Y., Buttstedt, A., Voisset, C., Galons, H., Blondel, M., Schwarz, E., Simonelig, M. (2011). Antiprion drugs 6-aminophenanthridine and guanabenz reduce PABPN1 toxicity and aggregation in oculopharyngeal muscular dystrophy. EMBO Molecular Medicine, 3(1), 35-49.
Beaumont, K., Webster, R., Gardner, I., Dack, K. (2003). Design of Ester Prodrugs to Enhance Oral Absorption of Poorly Permeable Compounds: Challenges to the Discovery Scientist. Current Drug Metabolism, 4(6), 461-485.
Ben-Shem, A., Garreau de Loubresse, N., Melnikov, S., Jenner, L., Yusupova, G., Yusupov, M. (2011). The Structure of the Eukaryotic Ribosome at 3.0 A Resolution. Science, 334(6062), 1524-1529.
Blondel, M., Soubigou, F., Evrard, J., Nguyen, P. hai, Hasin, N., Chedin, S., Reynald, G., Contesse, M. A., Friocourt, G., Stahl, G., Jones, G. W., Voisset, C. (2016). Protein Folding Activity of the Ribosome is involved in Yeast Prion Propagation. Scientific Reports, 6(1).
Castellani, R. J., Plascencia-Villa, G., Perry, G. (2019). The amyloid cascade and Alzheimer's disease therapeutics: theory versus observation. Laboratory Investigation.
Chartier, A., Benoit, B., Simonelig, M. (2006) A Drosophila model of oculopharyngeal muscular dystrophy reveals intrinsic toxicity of PABPN1. Embo J 25: 2253-2262
Chartier, A., Klein, P., Pierson, S., Barbezier, N., Gidaro, T., Casas, F., Carberry, S., Dowling, P., Maynadier, L., Bellec, M., Oloko, M., Jardel, C., Moritz, B., Dickson, G., Mouly, V., Ohlendieck, K., Butler-Browne, G., Trollet, C., Simonelig, M. (2015) Mitochondrial dysfunction reveals the role of mRNA poly(A) tail regulation in oculopharyngeal muscular dystrophy pathogenesis. PLoS Genet 11: e1005092
Das, D., Das, A., Samanta, D., Ghosh, J., Dasgupta, S., Bhattacharya, A., Basu, A., Sanya, S., Das Gupta, C. (2008). Role of the ribosome in protein folding. Biotechnology Journal, 3(8), 999-1009.
Ettmayer, P., Amidon, G. L., Clement, B., Testa, B. (2004). Lessons Learned from Marketed and Investigational Prodrugs. Journal of Medicinal Chemistry, 47(10), 2393-2404.
Falsig, J., Aguzzi, A. (2008) The prion organotypic slice culture assay—POSCA. Nat Protoc 3(4):555-562.
Harish, P., Dickson, G., Malerba, A. (2018). Advances in emerging therapeutics for oculopharyngeal muscular dystrophy. Expert Opinion on Orphan Drugs.
Hasin, N., Cusack, S. A., Ali, S. S., Fitzpatrick, D. A., Jones G. W. (2014) Global transcript and phenotypic analysis of yeast cells expressing Ssa1, Ssa2, Ssa3 or Ssa4 as sole source of cytosolic Hsp70-Ssa chaperone activity. BMC Genomics 15:194.
Heimbach, T., Oh, D.-M., Li, L. Y., Rodriguez-Hornedo, N., Garcia, G., Fleisher, D. (2003). Enzyme-mediated precipitation of parent drugs from their phosphate prodrugs. International Journal of Pharmaceutics, 261(1-2), 81-92.
Higuchi, T., Stella, V. J. (1975) Pro-drugs as Novel Drug Delivery System, ACS Sympos American Chemical Society, Washington, D.C.
Iida, M., Mashima, T., Yamaoki, Y., So, M., Nagata, T., Katahira, M. (2019). The anti-prion RNA aptamer R12 disrupts the Alzheimer's disease-related complex between prion and amyloid β. The FEBS Journal. doi:10.1111/febs.14819
Karapetyan, Y. E., Sferrazza, G. F., Zhou, M., Ottenberg, G., Spicer, T., Chase, P., Fallahi, M., Hodder, P., Weissmann, C., Lasmezas, C. I. “Unique drug screening approach for prion diseases identifies tacrolimus and astemizole as antiprion agents”, Proc Natl Acad Sci USA., vol. 110, no 17,2013, p. 7044-9.)
Langevin, C., Andreoletti, O., Le Dur, A., Laude, H., Beringue, V. (2011) Marked influence of the route of infection on prion strain apparent phenotype in a scrapie transgenic mouse model. Neurobiol Dis 41(1):219-225
Le Dur, A., Beringue, V., Andreoletti, O., Reine, F., Lai, T. L., Baron, T., Bratberg, B., Vilotte, J. L., Sarradin, P., Benestad, S. L., Laude, H. (2005). A newly identified type of scrapie agent can naturally infect sheep with resistant PrP genotypes. Proceedings of the National Academy of Sciences, 102(44), 16031-16036.
Malerba, A., Roth, F., Harish, P., Dhiab, J., Lu-Nguyen, N., Cappellari, O., Jarmin, S., Mahoudeau, A., Ythier, V., Laine, J., Negroni, E., Abgueguen, E., Simonelig, M., Guedat, P., Mouly, V., Butler-Browne, G., Voisset, C., Dickson, G., Trollet, C. (2019). Pharmacological modulation of the ER stress response ameliorates oculopharyngeal muscular dystrophy. Human Molecular Genetics.
Nguyen, P. hai Implication de l′activite chaperon de proteines du ribosome (PFAR) dans les mecanismes de prionization; identification de nouvelles molecules antiprion [Involvement of the chaperone protein activity of ribosome (PFAR) in prionization mechanisms; identification of novel antiprion molecules]. Thesis dissertation. Medecine humaine and pathologie. Universite de Bretagne occidentale—Brest, 2013. French. NNT: 2013BRES0026. HAL Id: tel-01434507
Nguyen, P. hai, Hammoud, H., Halliez, S., Pang, Y., Evrard, J., Schmitt, M., Oumata, N., Bourguignon, J. J., Sanyal, S., Beringue, V., Blondel, M., Bihel, F., Voisset, C. (2014a) Structure-activity relationship study around guanabenz identifies two derivatives retaining antiprion activity but having lost alpha2-adrenergic receptor agonistic activity. ACS Chem Neurosci 5(10):1075-1082
Nguyen, P. hai, Oumata, N., Soubigou, F., Evrard, J., Desban, N., Lemoine, P., Bouazizd, S., Blondel, M., Voisset, C. (2014b). Evaluation of the antiprion activity of 6-aminophenanthridines and related heterocycles. European Journal of Medicinal Chemistry, 82,363-371.
Oumata, N., Nguyen, P. hai, Beringue, V., Soubigou, F., Pang, Y., Desban, N., Massacrier, C., Morel, Y., Paturel, C., Contesse, M. A., Serge Bouaziz, S., Sanyal, S., Galons, H., Blondel, M., Voisset, C. (2013). The Toll-Like Receptor Agonist Imiquimod Is Active against Prions. PLoS ONE, 8(8), e72112.
Pang, Y., Kurella, S., Voisset, C., Samanta, D., Banerjee, D., Schabe, A., Das Gupta, C., Galons, H., Blondel, M., Sanyal, S. (2013). The Antiprion Compound 6-Aminophenanthridine Inhibits the Protein Folding Activity of the Ribosome by Direct Competition. Journal of Biological Chemistry, 288(26), 19081-19089.
Parsll D A, Kowal A S, Lindquist S (1994) Saccharomyces cerevisiae Hsp104 protein. Purification and characterization of ATP-induced structural changes. J Biol Chem 269(6):4480-4487
Pezron, I., Mitra, A. K., Duwuri, S., Tirucherai, G. S. (2002) Prodrug strategies in nasal drug delivery. Expert Opin. Ther. Pat. 12,331-340.
Raz, V., Routledge, S., Venema, A., Buijze, H., van der Wal, E., Anvar, S., Straasheijm, K., Klooster, R., Antoniou, M., van der Maarel, S. M. (2011). Modeling Oculopharyngeal Muscular Dystrophy in Myotube Cultures Reveals Reduced Accumulation of Soluble Mutant PABPN1 Protein. The American Journal of Pathology, 179(4), 1988-2000.
Reis, S. D., Pang, Y., Vishnu, N., Voisset, C., Galons, H., Blondel, M., Sanyal, S. (2011). Mode of action of the antiprion drugs 6AP and GA on ribosome assisted protein folding. Biochimie, 93(6), 1047-1054.
Steffansen, B., Nielsen, C. U., Brodin, B., Eriksson, A. H., Andersen, R., Frokjaer, S. (2004). Intestinal solute carriers: an overview of trends and strategies for improving oral drug absorption. European Journal of Pharmaceutical Sciences, 21(1), 3-16.
Stella, V. J. (2004) Prodrugs as therapeutics. Expert Opin. Ther. Pat. 14,277-280.
Stella, V. J. (2007) Prodrugs: challenges and rewards. (A. Press and Springer, eds.) Springer Singapore Pte. Limited, New-York.
Stella, V. J., Nti-Addae, K. W. (2007) Prodrug strategies to overcome poor water solubility. Adv. Drug Deliv. Rev. 59,677-94.
Tribouillard-Tanvier, D., Beringue, V., Desban, N., Gug, F., Bach, S., Voisset, C., Galons, H., Laude, H., Vilette, D., Blondel, M. (2008a). Antihypertensive Drug Guanabenz Is Active In Vivo against both Yeast and Mammalian Prions. PLoS ONE, 3(4), e1981.
Tribouillard-Tanvier, D., Dos Reis, S., Gug, F., Voisset, C., Beringue, V., Sabate, R., Kikovska, E., Talarek, N., Bach, S., Huang, C., Desban, N., Saupe, S. J., Supattapone, S., Thuret, J.-Y., Chedin, S., Vilette, D., Galons, H., Sanyal, S., Blondel, M. (2008b). Protein Folding Activity of Ribosomal RNA Is a Selective Target of Two Unrelated Antiprion Drugs. PLoS ONE, 3(5), e2174.
Vaccaro, A., Patten, S. A., Aggad, D., Julien, C., Maios, C., Kabashi, E., Drapeau, P., Parker, J. A. (2013). Pharmacological reduction of ER stress protects against TDP-43 neuronal toxicity in vivo. Neurobiology of Disease, 55,64-75.
Vilotte, J.-L., Soulier, S., Essalmani, R., Stinnakre, M.-G., Vaiman, D., Lepourry, L., Costa Da Silva, J., Besnard, N., Dawson, M., Buschmann, A., Groschup, M., Petit, S., Madelaine, M.-F., Rakatobe, S., Le Dur, A., Vilette, D., Laude, H. (2001) Markedly increased susceptibility to natural sheep scrapie of transgenic mice expressing ovine prp. J Virol 75(13):5977-5984
Wermuth, C. G. (2003) Designing prodrugs and bioprecursors. In The Practice of Medicinal Chemistry (Hardbound, ed), 2nd ed., pp. 561-585. Academic Press.
Wermuth, C. G. (2004). Selective Optimization of Side Activities: Another Way for Drug Discovery. Journal of Medicinal Chemistry, 47(6), 1303-1314.
Yang, C. Y., Dantzig, A. H., Pidgeon, C. (1999). Pharmaceutical Research, 16(9), 1331-1343.

1-15. (canceled)

16. A method of treating a proteinopathy comprising the administration of a composition comprising at least one compound selected from ebastine, azelastine, duloxetine, atomoxetine, benzydamine, biperiden, chloropyramine, citalopram, dicyclomine, nefopam, orphenadrine, prenylamine, triflupromazine and zimelidine, or one of the pharmaceutically acceptable salts, hydrates, isomers and racemates thereof to a subject in need of treatment.

17. The method according to claim 16, the composition comprising ebastine, or one of the pharmaceutically acceptable salts, hydrates, isomers and racemates thereof.

18. The method according to claim 16, the composition comprising azelastine, or one of the pharmaceutically acceptable salts, hydrates, isomers and racemates thereof.

19. The method according to claim 16, the composition comprising duloxetine, or one of the pharmaceutically acceptable salts, hydrates, isomers and racemates thereof.

20. The method according to claim 16, the composition further comprising at least one different compound selected from the group consisting of flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof.

21. The method according to claim 16, the composition comprising one of the following combinations: ebastine and flunarizine, ebastine and azelastine, ebastine and loperamide, azelastine and flunarizine, azelastine and loperamide, or flunarizine and loperamide, or the pharmaceutically acceptable salts, hydrates, isomers and racemates thereof.

22. The method according to claim 16, wherein said proteinopathy is selected from the group consisting of Creutzfeldt-Jakob disease, Gerstmann-Straussler-Scheinker syndrome, fatal familial insomnia, kuru, VPSPr disease, Lewy body disease, Parkinson's disease, Alzheimer's disease, Huntington's disease, amyotrophic lateral sclerosis, frontotemporal dementia, type 2 diabetes, oculopharyngeal muscular dystrophy, bovine spongiform encephalopathy, scrapie, chronic wasting disease of cervids, feline spongiform encephalopathy, camel spongiform encephalopathy and exotic ungulate encephalopathy.

23. The method according to claim 16, wherein said proteinopathy is linked to the accumulation of prion proteins PrP in the form of aggregates.

24. The method according to claim 23, wherein said proteinopathy is selected from the group consisting of Creutzfeldt-Jakob disease, Gerstmann-Straussler-Scheinker syndrome, fatal familial insomnia, kuru and VPSPr disease.

25. The method according to claim 22, wherein the proteinopathy is Creutzfeldt-Jakob disease and the composition comprises one of the following combinations: ebastine and flunarizine, ebastine and azelastine, ebastine and loperamide, azelastine and flunarizine, azelastine and loperamide, or flunarizine and loperamide, or the pharmaceutically acceptable salts, hydrates, isomers and racemates thereof.

26. method of inhibiting protein folding activity of ribosomes (PFAR) comprising the administration of a composition according to claim 16 to a subject in need of PFAR inhibition.

27. A composition comprising at least two different compounds, said at least two different compounds being:
a) at least one compound selected from ebastine, azelastine, duloxetine, atomoxetine, benzydamine, biperiden, chloropyramine, citalopram, dicyclomine, nefopam, orphenadrine, prenylamine, triflupromazine and zimelidine, or one of the pharmaceutically acceptable salts, hydrates, isomers and racemates thereof; and
at least one different compound selected from flunarizine, loperamide, ebastine, azelastine, metixene, guanabenz, 6-aminophenanthridine, imiquimod, tacrolimus, astemizole, doxycycline, amitriptyline, atomoxetine, benzydamine, biperiden, chloropyramine, chlorpromazine, citalopram, clemastine, clomipramine, desipramine, desloratadine, dicyclomine, diphenhydramine, doxepin, duloxetine, fluoxetine, haloperidol, imipramine, nefopam, orphenadrine, prenylamine, quinacrine (mepacrine), reboxetine, thioridazine, trifluoperazine, triflupromazine, alimemazine (trimeprazine) and zimelidine, or one of the pharmaceutically acceptable salts, hydrates, isomers and racemates thereof; or
b) one of the following combinations: ebastine and flunarizine, ebastine and azelastine, ebastine and loperamide, azelastine and flunarizine, azelastine and loperamide, or flunarizine and loperamide, or the pharmaceutically acceptable salts, hydrates, isomers and racemates thereof.

28. The composition according to claim 27, said composition further comprising one or more pharmaceutically acceptable carriers or excipients.

29. A method of inhibiting PFAR comprising contacting a ribosome in vitro or ex vivo with a composition comprising at least one compound selected from ebastine, azelastine, duloxetine, atomoxetine, benzydamine, biperiden, chloropyramine, citalopram, dicyclomine, nefopam, orphenadrine, prenylamine, triflupromazine and zimelidine, or one of the pharmaceutically acceptable salts, hydrates, isomers and racemates thereof and alimemazine (trimeprazine), amitriptyline, astemizole, clemastine, clomipramine, desipramine, desloratadine, diphenhydramine, doxepin, fluoxetine, haloperidol, imipramine, loperamide, reboxetine, thioridazine, trifluoperazine, chlorpromazine, and quinacrine (mepacrine), one of the pharmaceutically acceptable salts, hydrates, isomers and racemates thereof, or any combination thereof.
